(19) United States
(12) Patent Application Publication
(10) Pub. No.: US 2008/0044438 A1
(43) Pub. Date: Feb. 21, 2008

(54) Yeast Cell Particles As Oral Delivery Vehicles For Antigens

(76) Inventors: Gary R. Ostroff, Worcester, MA (US); Donald J. Tipper, Northborough, MA (US)

Correspondence Address: FOLEY HOAG, LLP;PATENT GROUP, WORLD TRADE CENTER WEST, 155 SEAPORT BLVD, BOSTON, MA, 02110, US

(21) Appl. No.: 11/687,368

(22) Filed: Mar. 16, 2007

Related U.S. Application Data
(60) Provisional application No. 60/783,493, filed on Mar. 17, 2006.

(51) Int. Cl. A61K 39/00; C12N 1/00; C12N 15/00; C12N 15/11; C12N 15/87; C12N 7/00

(52) U.S. Cl. 424/209.1; 424/246.1; 424/275.1; 424/279.1; 435/235.1; 435/254.21; 435/255.1; 435/255.2; 435/256.8; 435/320.1; 435/471; 536/23.4; 536/23.7; 536/23.72

(57) ABSTRACT

Provided herein are yeast cell particles (YCPs) comprising an antigen for use, e.g., as an oral, inhalation, mucosal or parenteral delivery vehicle for the antigen. A YCP may be obtained from a yeast cell by a process that removes at least some of the mannan from the outer cell wall layer, thereby exposing at least some of the cell wall β-1,3-glucan. The antigen may be expressed in the form of a fusion of the protein antigen to a scaffolding protein sequence that will allow the antigen to aggregate in the yeast cytoplasm. Exemplary scaffolds include proteins, e.g., viral capsid proteins that assemble into virus-like particles in yeast cytoplasm and proteins or peptides that self-aggregate.

### RELATED APPLICATIONS

This application claims the benefit of priority to U.S. provisional application 60/783,493 filed Mar. 17, 2006, which is herein incorporated by reference in its entirety.

### BACKGROUND

Vaccination remains the major mechanism of defense against well-recognized viral diseases, such as polio, measles, and influenza, and the best hope for eventually curtailing the continuing human immunodeficiency virus (HIV) crisis and protecting against newly recognized viral agents, such as severe acute respiratory syndrome (SARS). Vaccines are also routinely used to counter specific bacterial infections and are being actively developed for protection against major protozoal diseases, such as leishmaniasis and malaria. In addition, immunotherapy directed at tumor-specific antigens has become a routine aspect of cancer therapy.

Whereas some of the most effective anti-viral vaccine agents are attenuated viruses, capable of stimulating a tissue-appropriate immune response through the establishment of a transient infection, recombinant protein vaccines are widely used for protection against Hepatitis B (Tregnaghi et al. (2004) Rev. Panam. Salud. Publica. 15:35) and are being actively developed for treatment of the human papilloma viruses responsible for cervical cancer (e.g., HPV 16, 18, 45, 31), for protozoal infections, such as leishmaniasis (Ghosh et al. (2003) Mol. Cell. Biochem. 253:199 and Mazumdar et al. (2004) Vaccine 22: 1162) and for treatment of specific types of human cancer (e.g., www. ImmuneMedicine.com; Hotez et al. (2003) Int. J. Parasitol. 33:1245). Recombinant vaccines, in either DNA or protein form, are being vigorously developed for protection against biowarfare agents such as anthrax and plague.

Major factors affecting the utility of any vaccine are efficacy, safety and, or course, cost. For example, although the World Health Organization (WHO) advocates widespread childhood vaccination against hepatitis B, and effective injectable recombinant vaccines are available (Tregnaghi et al., supra), they cost too much for use in countries such as India (Sahni et al. (2004) Indian J. Gastroenterol. 23:16). African countries are even less able to sustain the cost. Further, the need to produce these vaccines in developed countries and to maintain refrigeration until vaccine administration (the cold chain) is not practical in the developing world. Finally, in third world countries, the necessity for re-use of needles clearly increases the risk of HIV transmission.

### SUMMARY

Provided herein are yeast cell particles (YCPs) comprising an antigen for use, e.g., as an oral, inhalation, mucosal or parenteral delivery vehicle for the antigen. An antigen may be a heterologous antigen, i.e., an antigen that is not normally expressed (in that form) in the yeast cell from which the YCP was prepared. A YCP may be obtained from a yeast cell of any species, including both wild-type and mutant strains, for example with alterations in cell wall composition, by a process that removes at least some of the mannan from the outer cell wall layer, thereby exposing at least some of the cell wall β-1,3-glucan. A YCP may be a yeast cell that comprises less mannan in its cell wall relative to the amount of mannan prior to the processing to remove the mannan and/or a yeast cell that comprises less mannan relative to a wild-type yeast, e.g., a wild-type yeast of the same strain. The antigen may be expressed in the yeast cell prior to processing of the yeast cell to form a YCP, and is expressed in a form that allows the antigen to be retained during processing of the yeast cell. For example, the antigen may be expressed in the form of a fusion of the protein antigen to a scaffolding protein sequence that will allow the antigen to aggregate in the yeast cytoplasm. Exemplary scaffolds include proteins, e.g., viral capsid proteins that assemble into virus-like particles in yeast cytoplasm and proteins or peptides that self-aggregate.

Provided herein are yeast cell particles (YCPs) having a reduced amount of mannan in their cell wall relative to that of a wild-type yeast, wherein the YCPs comprise a heterologous antigen. A YCP may have a sufficient amount of mannan removed to expose the underlying beta 1,3-glucan to allow it to interact with an M cell of the gastrointestinal tract of a eukaryote. A YCP may have about 10-50% of mannan removed.

The antigen may be linked to a scaffold that allows the antigen to form an aggregate in the cytoplasm of a yeast cell. For example, the scaffold may be a protein that forms virus-like particles (VLPs). The scaffold may be a VP1 capsid protein of mouse polyoma virus or a functional homolog thereof. The scaffold may comprise SEQ ID NO: 20. The scaffold may also be a Hepatitis B surface antigen (HBsAg) or a functional homolog thereof. The scaffold may comprise SEQ ID NO: 18. The scaffold may be a non-pathogenic protein that self-aggregates in the cytoplasm of a yeast cell or a functional homolog thereof. The scaffold may be a non-pathogenic protein of yeast. The scaffold may be a self-aggregating N-terminal portion of the yeast Ure2 protein or a functional homolog thereof, such as comprising SEQ ID NO: 22.

The antigen and the scaffold may be linked through a linker, such as a flexible peptide linker. A linker may comprise about 5-10 amino acids, e.g., the amino acid sequence GGSSGGSS (SEQ ID NO: 23).

The antigen may be a protein from a pathogen or a functional homolog thereof. The antigen may be selected from the group consisting of an LcrV protein from Yersinia pestis, a protective antigen (PA) from B. anthracis, hemagglutinin (HA) from influenza H5 and functional homologs thereof. The yeast may be Saccharomyces cerevisiae.

Also provided are compositions comprising a YCP and a pharmaceutically acceptable carrier or vehicle. A composition may be a vaccine preparation.

Also described herein are nucleic acids comprising a nucleotide sequence encoding a fusion protein comprising an antigen and a scaffold that allows the antigen to form an aggregate in the cytoplasm of a yeast cell, wherein the nucleotide sequence encoding the fusion protein is operably linked to a promoter that is transcriptionally active in yeast. The antigen may be an antigen from a pathogen or a functional homolog thereof and the scaffold is a protein that forms VLPs, a non-pathogenic protein that self-aggregates in the cytoplasm of a yeast cell or a functional homolog thereof. The scaffold may be a self-aggregating N-terminal portion of the yeast Ure2 protein or a functional homolog thereof. The antigen may be selected from the group consisting of an LcrV protein from Yersinia pestis, a protective antigen (PA) from B. anthracis, hemagglutinin (HA) from influenza H5 and functional homologs thereof. The nucleic acid may be in a vector, e.g., an expression vector. Another embodiment includes yeast cells, e.g., S. cerevisiae yeast cells, comprising a nucleic acid described herein.

Methods are also encompassed. For example, a method for preparing a yeast cell may comprise (i) providing a yeast cell comprising a heterologous antigen as an insoluble aggregate; and (ii) subjecting the yeast cell to a treatment allowing sufficient removal of mannan from its outer cell wall layer to expose the underlying beta 1,3-glucan and allow it to interact with an M cell of the gastrointestinal tract of a eukaryote. Step (ii) may comprise incubating the yeast cell in a solution having a pH of about 10-13 at about 40-50° C. for about 5 to 10 minutes. The method may further comprise neutralizing the solution after step (ii). A method may comprise cultivating a yeast cell comprising a nucleic acid encoding a fusion protein comprising the heterologous antigen fused to a scaffold that allows the antigen to form an aggregate in the cytoplasm of the yeast cell, under conditions in which the yeast cell expresses the fusion protein; and (ii) subjecting the yeast cell to a treatment allowing sufficient removal of mannan from its outer cell wall layer to expose the underlying beta 1,3-glucan and allow it to interact with an M cell of the gatrointestinal tract of a eukaryote. Step (i) may be preceded by a step in which the nucleic acid of step (i) is introduced into the yeast cell.

A method for preparing a vaccine may comprise combining a YCP described herein with a pharmaceutically acceptable carrier.

A method for protecting a subject from an infection by a pathogen, may comprise administering to a subject in need thereof a therapeutically effective dose of a YCP described herein, wherein the antigen is a protein from the pathogen or a functional homolog thereof that triggers a protective immune response against the pathogen. The YCP may be administered orally. The method may be for protection against plague, anthrax or influenza, and the antigen may be selected from the group consisting of an LcrV protein from Yersinia pestis, a protective antigen (PA) from B. anthracis, hemagglutinin (HA) from influenza H5, respectively, and functional homologs thereof.

Also provided are methods for treating a subject who has or is likely to develop a hyperproliferative disease, comprising administering to a subject in need thereof a therapeutically effective dose of a YCP described herein, wherein the antigen is a hyperproliferative-associated protein or a functional homolog thereof that triggers an immune response against the cells that cause the hyperproliferative disease. Other methods include those for treating a subject who has or is likely to develop an autoimmune disease or allergy, comprising administering to a subject in need thereof a therapeutically effective dose of a YCP described herein, wherein the antigen is a protein associated with the autoimmune disease or allergy or a functional homolog thereof that triggers an immune response against the cells that cause the autoimmune disease or allergy. Also provided are kits, e.g., kits comprising one or more doses of YCPs described herein.

### BRIEF DESCRIPTION OF THE DRAWINGS

FIG. 1. Schematic diagram 100 of a transverse section of a yeast cell wall, showing, from outside to inside, an outer fibrillar layer 110, an outer mannoprotein layer 120, a beta glucan layer 130, a beta glucan-chitin layer 140, an inner mannoprotein layer 150, the plasma membrane 160 and the cytoplasm 170.

FIG. 2. The pG2-GFP vector and GFP fusion constructs expressed in it. The indicated relative levels of expression are derived from GFP fluorescence data and are the average from multiple cultures grown under optimal expression conditions. Other proteins expressed at similar levels include HBsAg and VP1 without the GFP tag.

FIG. 3: VP1-GFP YCP's (retaining 80% of initial mannan content) phagocytosed by 3T3-D1 cells. Light photomicrograph showing YCP's inside 3T3 cells.

FIG. 4. SDS-PAGE of total proteins from yeast cells and YCP's. A: Lanes 1 and 4, Western (α-VP1) and coomassie-stain; cells expressing VP1-GFP (70 kDa). Lane 5, YCP's made from these cells. Lanes 2 and 3, Western (α-VP1) and coomassie-stain; cells expressing VP1-GFP, showing both the 70 kDa monomer (lower band) and the presumed VP1-GFP pentamer (upper arrow). Lane 6, YCP's made from these cells. Lanes 7 and 8, coomassie-stain and Western (α-GFP): cells expressing U2N-GFP. B. Retention of GFP fusion proteins during YCP extraction. Lanes 1 and 15, Western; cells expressing U2N-GFP (α-GFP) and VP1-GFP (α-VP1), respectively. Lanes 2-14, Coomassie stain; each set of 4 lanes shows proteins from cells extracted for 0, 4, 8 and 12 minutes. Lanes 2-5, cells expressing U2N-GFP. Lanes 6-9, cells expressing GFP-VP1. Lanes 10-13, cells expressing VP1-GFP. Lane 14, purified GST-GFP (58 kDa).

FIG. 5. Yeast expressing VP1-GFP. C1-3: ConA-594-stained cells after increasing time of extraction retaining, respectively, 100, 80 and 20% mannan, G1-3: GFP fluorescence in different fields of the same cell samples as C1-3.

FIG. 6. Composite micrographs showing both ConA-594-stained cell walls and GFP aggregates in A, cells expressing VP1-GFP. B, YCP's derived from those cells. C, cells expressing U2N-GFP and D, YCP's derived from those cells.

FIG. 7. Electronmicrographs of negatively stained high speed pellet fractions from cells expressing VP1-GFP VLPs (A), GFP-VP1 VLPs (B) and U2N-GFP fibrils (C).

FIG. 8. Micrograph of YCP VP1-GFP ingested by 3T3-D1 cells. A—light photomicrograph, B—fluorescent photomicrograph visualizing GFP. C—fluorescent photomicrograph visualizing Congo Red binding to beta 1,3-glucan in YCP walls.

FIG. 9. FACS analysis of beta glucan exposure in cells and YCP's. Binding of mouse monoclonal antibody specific for beta-1,3 glucan (Mikle et al.; Wheeler et al.) is detected using goat anti-mouse IgG labeled with phycoerythrin. The curves represent untreated cells expressing Apo A1 (1 unTx, peak at 2-8), the YCP's produced from these cells under standard conditions (5 Apo 2-3, peak at 200-500) and YGMP particles (2 YGMP peak at 200-2000). YGMP particles are derived from yeast cell walls by vigorous alkali extraction.

FIG. 10. Serum IgG responses to sub-cutaneous doses of YCP's expressing U2N-GFP. ELISA data for groups of 4 mice receiving the indicated vaccine preparations after a prime and boost dose. 11.5 is a preparation of intact YCP's extracted at pH 11.5. 118. is a preparation of intact YCP's extracted at pH 11.8. 11.8. broken is the same 11.8 YCP's after bead breakage. 11.8 50/50 is a mixture of equal amounts of broken and intact 11.8 YCP's. Results are shown as 1n2 of dilutions giving average responses at least 2 fold over background.

FIG. 11. Serum IgG responses on day 49 after the primary vaccination and 10 days after the second boost with a YCP VP1-GFP vaccine. ELISA data are shown at 1/200 dilution for (1), ip doses and (2), oral doses. A: responses to GFP. B: responses to VP1. Results are shown as antisera dilutions giving average responses at least 2 fold over background.

### DETAILED DESCRIPTION

Yeast Cell Particles (YCPs) and their Preparation

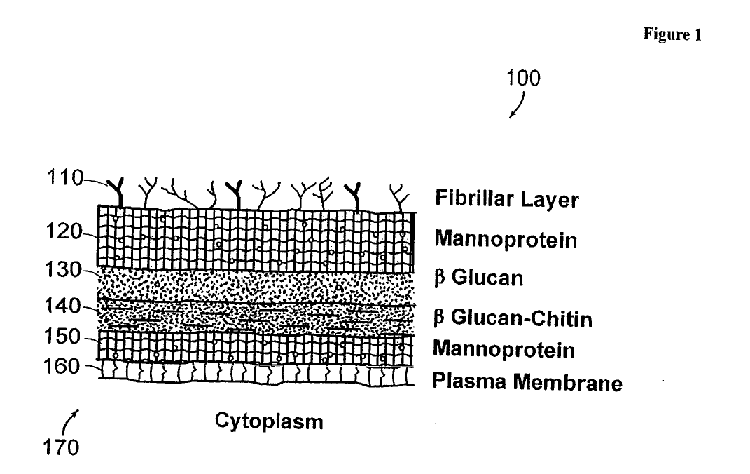

In one embodiment, a yeast cell is processed under conditions that partially remove the outer cell wall layer mannan and optionally the inner cell wall layer mannan, thereby exposing at least some of the cell wall β-1,3-glucan (see FIG. 1 for a schematic of a yeast cell wall). The procedure preferably allows retention of a significant amount of a recombinant protein expressed in the yeast cell in an aggregated form. The total amount of mannan that remains in the yeast particle after treatment may be at least about 10%, 20%, 30%, 40%, 50%, 60%, 70%, 80% or 90% relative to the total amount of mannan present in the yeast cell prior to its processing or relative to that of a wild-type yeast. In certain embodiments, the total amount of mannan that is retained on a yeast particle is about 10-30%, 20-40%, 30-50%, 40-60%, 50-70%, 60-80% or about 70-90% relative to the total amount of mannan present in the yeast cell prior to its processing or relative to that of a wild-type yeast. In certain embodiments, the total amount of mannan that is removed from the yeast cell is about 10%, 20%, 30%, 40%, 50%, 60%, 70%, 80%, 90%, 10-30%, 20-40%, 30-50%, 40-60%, 50-70%, 60-80% or about 70-90%. In certain embodiments, mannan may be removed essentially only from the outer cell wall mannan layer. The same amounts of mannan may be removed from or retained in the outer cell wall mannan layer as described above for total mannan content.

Without wanting to be restricted to a particular mechanism of action, it is believed that the exposed glucan interacts with M cell surface receptors resulting in efficient engulfment and transport of the yeast cell particle to local antigen presenting cells (APCs), e.g., the dendritic cells (DC's) and macrophages (MP) in the gastrointestinal (G1) tract Peyer's patches. DC's should also interact directly with YCP's using extensions projecting into the GI tract lumen between epithelial cells. The glucan and mannan cell wall components are potent adjuvants, and will stimulate APCs to mature and migrate to the lymphatic system where they interact with T cells, presenting the antigen expressed in the YCPs and initiating the immune response. Glucan and mannan differentially bias the response towards T helper (TH)₂ and THI responses, respectively (see, e.g., Breinig et al. (2003) FEMS Immunol. Med. Microbiol. 38:231; Brown et al. (2003) Immunity 19: 311; Hong et al. (2003) Cancer Res. 63:9023 and Stubbs et al. (2001) Nat. Med. 7:625).

In certain embodiments, at least about 10%, 20%, 30%, 40%, 50%, 60%, 70%, 80%, 90%, 95% 99%, 10-30%, 20-40%, 30-50%, 40-60%, 50-70%, 60-80% or about 70-90% of glucan is retained.

Processing of yeast cells to obtain YCPs retaining at least some of their cell wall mannan includes chemical, thermal and enzymatic methods. An exemplary method may comprise one or more of the following steps, not necessarily in the order provided: (i) providing a suspension of yeast cells, e.g., at about 10⁷ to 10⁹ yeast cells/ml; (ii) bringing the pH of the suspension of yeast cells to about pH 11-13, e.g., about pH 12-13, or about pH 12.10; (iii) increasing the temperature of the yeast cell suspension, e.g., by immersing the container comprising the yeast cell suspension in a bath of warm-hot water, such as at a temperature of about 40-60° C., e.g., 45-50° C.; (iv) stirring the yeast cell suspension while it is incubated in the warm-hot water of step (iii); (v) adding NaOH to maintain the pH at about 11-13, e.g., at 11 or 11.85, with compensation for the effect of temperature on pH; (vi) maintaining the yeast cell suspension at this temperature for about 1 to about 20 minutes, e.g., 15, 8, 5 or 2 minutes, so as to obtain the desired amount of mannan removal and mannan retained in the yeast cells; and (vii) stopping the reaction, e.g., by the addition of a buffer to bring the pH to about 6-8.

An exemplary method is as follows. Yeast cells at 5.10⁸/ml, at 25° C. in the absence of buffer, are adjusted to pH 12.10 with NaOH, and then immersed, with stirring, in a bath at 48 to 50° C. Cell suspension temperature reaches 43-45° C. within 5-6 min, and pH starts to drop sharply after 6-7 min, as the cells become fully permeable to the alkali. Additional NaOH is added to keep the pH at 11.85 (equivalent to pH 12 at 25° C.). Maintenance at pH 11.85 requires NaOH addition at 30-60 second intervals for the following 3 minutes, but after that pH is nearly stable. One can use an automatic pH stat, which is particularly useful for large scale vaccine production. The reaction is stopped by addition of sufficient 1M Tris/HCl to bring the pH down to 7.5.

Another example is as follows: yeast cells at 2.10⁹/ml, are equilibrated at 45° C. in the absence of buffer, and are then adjusted to pH 11.8 (equivalent to pH 12 at 25° C.) with NaOH. The cell suspension at 45° C. is maintained at pH 11.8 by the frequent addition of additional NaOH. After 5-6 min, the pH is nearly stable. One can use an automatic pH stat, which is particularly useful for large scale vaccine production. The reaction is stopped after 10-15 min by addition of sufficient 1M Tris/HCl to bring the pH down to 7.5 and the YCP's are immediately washed extensively in PBS at 4° C.

In the above protocols, the following variations can be made: the cell concentration can be anything up to 5.10⁹ cells/ml; the temperature can be up to 55° C.; the pH can be between 11.5 and 12.2; and the duration can be between 2 to 20 minutes. The order of events can also be modified, e.g., the cells may be heated first or the pH may be adjusted first. The two above procedures are representative of these variations.

The protocols can be adapted to obtain YCPs containing various amounts of mannan. For example, varying the temperature and duration of the reaction will affect the amount of mannan retained in YCPs. Using the above protocol, it takes about 5-7 minutes to remove 10-20% of mannan and about 10-12 minutes to remove 40-60% mannan.

Processing of recombinant yeast cells into yeast cell particles may also remove at least some of, e.g., about 10%, 30%, 50%, 70%, of the soluble yeast cell proteins, while retaining all or most of the scaffolded antigen protein. For removing additional non-antigen yeast cell proteins, YCPs may be suspended at 25° C. in a non-ionic detergent, such as 1% triton X100 with mixing at 1 min intervals for 2-10 min, then centrifuged and washed thoroughly in PBS.

Other methods of mannan removal include: partial autolysis using methods previously described to produce yeast extracts, pH 8/EDTA extraction, surfactant (i.e. SDS) extraction, aqueous/organic solvent extraction, protease digestion of intact cells to remove extracellular mannoproteins, glycosidase (mannosidase and other endoglycosidase) digestion of mannan oligosaccharides, heat extraction, acid extraction, etc. Methods using EDTA and SDS are described, e.g., in Ruiz-Herrera (1994) Microbiology 140:1513 and Casanova et al. (1991) J. Gen. Microbiol. 137:1045. Methods using proteases are described, e.g., in Zlotnik et al. (1984) J. Bacteriol. 159:1018. Methods using hot water are described, e.g., in Shibata et al. (1984) Microbiol. Immunol. 28:1283.

In yet another embodiment, a mutant yeast strain that is deficient in cell surface mannan because of defects in N-glycosylation (e.g., alg, mnn, ost, cwh or sec mutant strains, etc; Klis F M, Boorsma A, De Groot P W. Cell wall construction in Saccharomyces cerevisiae. Yeast. 2006 February; 23(3): 185-202) may be used. Such strains have less mannan than the corresponding wild-type yeast strain.

Although the mannan in the yeast cell wall is essentially in the form of mannoprotein, it is not necessary to remove the protein that is associated with the mannan. Some methods for mannan removal, e.g., those described herein, may remove primarily mannan and not the associated protein or may remove the mannan and some or all of its associated protein.

The amount of mannan remaining on the YCPs may be determined by several methods. An exemplary method may use concanavalin A (ConA), which is a lectin that binds selectively to mannan. One method comprises staining YCPs with fluorescently labeled conA-alexafluor 594 or conA-alexafluor 488 and measuring the fluorescence by fluorimetric analysis. Another method comprises staining YCPs with fluorescently labeled conA-alexafluor 647 or conA-alexafluor 488 and measuring the fluorescence by FACS analysis. These ConA assays may be standardized by Dionex HPLC analysis of total mannose and glucose content in the YCP's.

The amount of β-glucan exposed in YCP's is a more direct measure of the potential efficiency of YCP uptake by dendritic cells (DC), macrophages and M-cells and, therefore, of efficacy of vaccine delivery and glucan-dependent adjuvant activity. The most quantitative assay is provided by fluorescence-based FACS analysis of binding of an anti-glucan monoclonal antibody (Wheeler et al.; Meikle et al.). Minimal binding of antibody (negative control) is defined using fresh yeast cells grown under conditions for antigen expression. Maximal binding of antibody (positive control) is defined using YGP particles, yeast cell walls stripped of all manno-protein by intense alkali extraction. The fluorescence signal strength for these negative and positive controls is 3-8 and 2000-5000. YGMP particles, yeast cell walls stripped of a large fraction of manno-protein by vigorous but less intense alkali extraction, give a broad signal from 200-3000. Functional YCP's have signals of 50-500, representing a 10 to 100 fold increase in antibody binding.

β-glucan-mediated binding and entry of YCPs into DCs is mediated by dectin-1 and TLR2 receptors and a more direct measure of uptake efficacy is provided by assay of uptake of YCPs by the murine 3T3 cell line expressing dectin-1 (47). Murine 3T3 cells not expressing the dectin-1 receptor are unable to take up yeast or YCPs. Expression of dectin-1 receptor protein is sufficient to allow uptake of cells dependent entirely upon exposed β-glucan.

Yeast strains that may be used include any yeast strain, provided that yeast cells from that strain can be processed to remove some of its cell wall mannan and retain at least some of its inner cell wall β-1,3-glucan. A yeast cell may belong to one of three classes of yeast: Ascomycetes, Basidiomycetes and Fungi Imperfecti. Exemplary genera of yeast strains that may be used include Saccharomyces, Candida, Cryptococcus, Hansenula, Kluyveromyces, Pichia, Rhodotorula, Schizosaccharomyces and Yarrowia. Exemplary species of yeast strains that may be used include Saccharomyces cerevisiae, Saccharomyces carlsbergensis, Candida albicans, Candida kefyr, Candida tropicalis, Cryptococcus laurentii, Cryptococcus neoformans, Hansenula anomala, Hansenula polymorpha, Kluyveromyces fragilis, Kluyveromyces lactis, Kluyveromyces marxianus var. lactis, Pichia pastoris, Pichia Hansenula, Rhodotorula rubra, Schizosaccharomyces pombe, and Yarrowia lipolytica. It is to be appreciated that a number of these species include a variety of subspecies, types, subtypes, etc. that are meant to be included within the aforementioned species. Useful yeast strains are those that are capable of replicating plasmids to a high copy number, such as a S. cerevisiae cir.zero strain (e.g., DB1, DS65, DS212, and DS569). A preferred yeast strain is one that has been designated by the U.S. Food and Drug Administration as being GRAS (i.e., Generally Recognized as Safe) for use in food products.

While oral doses should comprise intact YCP's, immune responses to parenterally administered YCP's may be enhanced by using a mixture of broken and unbroken YCP's. It is probable that initial parenteral doses should include unbroken YCP's to ensure co-delivery of scaffolded antigen and their yeast cell wall vehicle and adjuvant to the same phagocytic vesicle in APC's (Blander) while the efficacy of boost doses may be enhanced by using broken YCP's in which scaffolded antigen has direct access to B-cell receptors. Use of a mixture avoids the need for two distinct formulations. This mixture may comprise 20 to 90% broken YCP's by weight. A suspension of YCP's in PBS at 4 C (108 to 2.109/ml) is broken by vigorous mixing with an equal volume of 0.5 mM clean and sterile glass beads using any appropriate device such as a vortex mixer or a bead-beater. Breakage is monitored by microscopy, beads are removed, e.g., by filtration, and YCP fragments, comprising cell wall fragments, scaffolded antigen and other proteins, are recovered by centrifugation. Losses are less than 2%.

In one embodiment, a yeast cell is modified to express an antigen prior to processing it to obtain a YCP. For example, a yeast cell may be transformed with a nucleic acid, e.g., a plasmid or expression vector, encoding the antigen. The transformed yeast cells may then be grown under conditions allowing the expression of the antigen. YCPs may be prepared from these cells once they have reached the desired level of expression of the antigen.

A nucleic acid encoding an antigen may be operably linked to one or more regulatory sequences, e.g., a promoter. A variety of promoters may be used for expression of an antigen in a yeast cell. Preferred promoters are those that allow high levels of expression of the antigen in a yeast cell. A promoter may be constitutive or inducible. A preferred promoter is a tightly regulated inducible promoter such that a high copy number can be achieved in the effective absence of expression, avoiding selection for lower expression. Examples are the normally divergent GAL1p and GAL10p promoters that are tightly suppressed in glucose media and highly induced by galactose, once catabolite repression has been relieved by growth on a non-repressing carbon source such as lactate or glycerol. An open reading frame encoding an antigen may be inserted into a GAL1p vector (Cartwright et al. (1994) Yeast 10:497 and Harley et al. (1998) J. Biol. Chem. 273:24963). Other promoters and vectors that may be used include the hybrid GAL1-CYCp promoter in the Yep URA3 leu2d vector pPAP1488 in strain PAP1502 (Pedersen et al. (1996) J. Biol. Chem. 271:2514). This strain has plasmid pPAP1488 integrated at the trp1 locus. This provides an additional copy of the GAL4 gene driven by the GAL10 promoter, so that high levels of the Gal4p positive activator are produced when GAL expression is induced. Growth in the absence of uracil (Ura D/O medium) results in a vector copy number of 15-20, determined by 2 micron replication functions. The number of copies of the vector can further be increased, e.g., at least 10 fold, by culture of the yeast cells in media lacking leucine (Leu D/O), due to the very weak promoter associated with the defective leu2d allele. A proportional increase in GAL1p driven expression requires the high galactose-induced levels of the Gal4p activator provided in strain PAP1502 (Pedersen et al., supra) by the integrated PAP1488 plasmid. Any other ura3 leu2 Gal+S. cereviseae strain into which this plasmid is inserted may be used in place of strain PAP1502. Techniques that may be used include that described in Tipper and Harley (2002; Mol Biol of the Cell, 13: 1158-1174). For example, an internal fragment of the CAN1 gene (codons 91-410) may be inserted in pPAP1488. The unique restriction sites in this fragment may be used to target integration at CAN1, under selection for canavanine resistance. Strains such as CRY1 and CRY2, described in Tipper and Harley (supra) may be used for this purpose.

Levels of antigen produced may be further increased by either one or both of these vector modifications: insertion of the GAL1-GDH promoter (Bitter et al. (1988) Gene 69:193), and insertion of two copies of the PGK terminator (Cartwright et al., supra), producing vector pG2-GFP. Generally strong promoters and high copy number plasmids are preferable for expressing high levels of antigen in a yeast cell.

Another yeast promoter that may be used is the promoter of the glycerol-3-phosphate dehydrogenase gene (GPD 1). Expression of such polypeptides utilizing the GPD1 promoter can be regulated by the presence (repressed) or absence (deprepressed) of high levels of sucrose or glucose in the fermentation medium. Alternatively, a non-repressing carbon source, such as glycerol or ethanol, can be added to the fermentation medium (see U.S. Pat. No. 5,667,986).

Other promoters for expression in yeast include promoters of genes encoding the following yeast proteins: alcohol dehydrogenase I (ADH1) or II (ADH2), phosphoglycerate kinase (PGK), triose phosphate isomerase (TPI), glyceraldehyde-3-phosphate dehydrogenase (GAPDH; also referred to as TDH3, for triose phosphate dehydrogenase), galactose-1-phosphate uridyl-transferase (GAL7), UDP-galactose epimerase (GAL10), cytochrome c₁ (CYC1) and acid phosphatase (PHO5). Hybrid promoters, such as the ADH2/GAPDH, CYC1/GAL10 and the ADH2/GAPDH promoter, which is induced when glucose concentrations in the cell are low (e.g., about 0.1 to about 0.2 percent), may also be used. In S. pombe, suitable promoters include the thiamine-repressed nmt1 promoter and the constitutive cytomegalovirus promoter in pTL2M (Sasagawa et al, 2005). A person of skill in the art would understand that any yeast expression system can be used to produce sufficient amounts of antigen.

A number of upstream activation sequences (UASs), also referred to as enhancers, may be used in addition to a promoter. Exemplary upstream activation sequences for expression in yeast include the UASs of genes encoding the following proteins: CYC1, ADH2, GAL1, GAL7, GAL10 and ADH2, as well as other UASs activated by the GAL4 gene product.

Exemplary transcription termination sequences for expression in yeast include the termination sequences of the α-factor, GAPDH, CYC1 and PGK genes. One or more termination sequences may be used. As shown in the Examples, two termination sequences of the PGK gene provided a higher expression level of the recombinant protein, relative to the presence of a single termination sequence.

A YCP may comprise one or more antigens, which may be expressed from one or more nucleic acid sequences. For example, two or more antigens may be encoded by 2, 3, 4, 5 or more nucleic acid sequences. The antigens may each be expressed from a separate promoter. One or more antigens may also be linked together in a fusion protein, directly, or indirectly, e.g., through a linker, such as to preserve independent domain folding ensuring immune recognition of the antigens in their native folded forms.

An antigen present in a YCP may be identical to a naturally-occurring antigen, or it may be a functional homolog thereof, i.e. a homolog that provides the desired immune response against the naturally-occurring antigen. A functional homolog of an antigen may be a homolog having one or more epitopes of the naturally-occurring antigen in single or multiple copies. Functional homologs may be homologs that share a certain percentage identity in amino acid sequence with the naturally-occurring antigen and/or they may comprise only a portion of a naturally-occurring antigen.

In one embodiment, an antigen present in a YCP comprises an amino acid sequence that is at least about 60%, 70%, 80%, 90%, 95%, 98%, or 99% identical to an amino acid sequence of a naturally-occurring antigen, such as an antigen described herein, or a portion thereof. A portion of an antigen may consist of at least about 6, 10, 15, 20, 30, 40, 50 or 100 amino acids. An antigen present in a YCP may also be encoded by a nucleic acid that comprises a nucleotide sequence that is at least about 60%, 70%, 80%, 90%, 95%, 98%, or 99% identical to a nucleotide sequence encoding a naturally-occurring antigen or a portion thereof. A nucleic acid encoding an antigen may be modified to increase the level of expression in yeast without affecting the expressed amino acid sequence. For example, codons can be optimized to use those that are used more frequently in the particular yeast strain employed.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100). In one embodiment the two sequences are the same length.

The determination of percent identity between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al. (1997) Nucleic Acids Res. 25:3389-3402. Alternatively, PSI-Blast can be used to perform an iterated search which detects distant relationships between molecules. When utilizing BLAST, Gapped BLAST, and PSI-Blast programs, the default parameters of the respective programs (e.g., XBLAST and NBLAST) can be used. See http://www.ncbi.nlm.nih.gov. Another preferred, non-limiting example of a mathematical algorithm utilized for the comparison of sequences is the algorithm of Myers and Miller, (1988) Comput Appl Biosci, 4:11-7. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson and Lipman (1988) Proc. Natl. Acad. Sci. USA 85:2444-2448. When using the FASTA algorithm for comparing nucleotide or amino acid sequences, a PAM120 weight residue table can, for example, be used with a k-tuple value of 2.

The percent identity between two sequences can be determined using techniques similar to those described above, with or without allowing gaps. In calculating percent identity, only exact matches are counted.

An antigen present in a YCP may also be encoded by a nucleic acid that hybridizes under low, medium or high stringency hybridization conditions to a nucleic acid that encodes a naturally-occurring antigen or a portion thereof. An antigen that differs from a naturally-occurring antigen preferably induces an immune response to the naturally-occurring antigen and may share one or more epitopes with the naturally-occurring antigen.

Hybridizations may be conducted under any of the following conditions: high stringency conditions of 0.2 to 1×SSC at 65° C. followed by a wash at 0.2×SSC at 65° C.; low stringency conditions of 6×SSC at room temperature followed by a wash at 2×SSC at room temperature; hybridization conditions including 3×SSC at 40 or 50° C., followed by a wash in 1 or 2×SSC at 20, 30, 40, 50, 60, or 65° C. Hybridizations can be conducted in the presence of formaldehyde, e.g., 10%, 20%, 30% 40% or 50%, which further increases the stringency of hybridization. Theory and practice of nucleic acid hybridization is described, e.g., in S. Agrawal (ed.) Methods in Molecular Biology, volume 20; and Tijssen (1993) Laboratory Techniques in biochemistry and molecular biology-hybridization with nucleic acid probes, e.g., part I chapter 2 “Overview of principles of hybridization and the strategy of nucleic acid probe assays,” Elsevier, N.Y. provide a basic guide to nucleic acid hybridization.

An antigen that is expressed in a YCP may be a fusion or chimeric protein. For example, in addition to being linked to a scaffold, as further described below, an antigen may also be fused to one or more amino acids or to a heterologous peptide of at least about 3, 5, 10, 15, 20, 25, 30, 40, 50 or more amino acids. A heterologous polypeptide may be a polypeptide that allows easy detection of the polypeptide of interest. For example, a protein may be fused to a “Tag peptide” encoded by a “Tag sequence,” such as a hexahistidine tag, a myc-epitope (e.g., see Ellison et al. (1991) J Biol Chem 266:21150-21157) which may include a 10-residue sequence from c-myc, a peptide from the pFLAG system (International Biotechnologies, Inc.), a peptide from the pEZZ-protein A system (Pharmacia, N.J.), and a 16 amino acid portion of the Haemophilus influenza hemagglutinin protein. Furthermore, any peptide can be used as a Tag peptide so long as a reagent, e.g., an antibody interacting specifically with the Tag peptide, is available or can be prepared or identified. A heterologous peptide preferably does not interfere with the immune recognition of the antigen.

Other chimeric proteins include those comprising a fusion of the antigen to a peptide that enhances antigen presentation. For example, an antigen may be fused to the invariant chain (Ii) protein (protein that associates with major histocompatibility complex (MHC) molecules) or a fragment thereof (see, e.g., Gregers et al. (2003) Int. Immunol. 15:1291). Other fusion proteins may comprise a peptide that effects the direction of antigen presentation (Th1 vs Th2). Exemplary peptides include cholera toxin (CT) or the enzymatically inactive receptor-binding B subunit or CT (CTB) or portions thereof and CTA1 or an enzymatically inactive mutant CTA1R7K (Lycke N. (2005) Curr. Mol. Med. 5:591).

Nucleic acids, e.g., expression vectors, can be introduced into yeast cells according to methods known in the art, e.g., by transfection, electroporation, microinjection, lipofectin, adsorption, and protoplast fusion. Transformed nucleic acid molecules can be integrated into a yeast chromosome or maintained on extrachromosomal vectors using techniques known to those skilled in the art.

Effective conditions for the production of YCPs comprising an antigen include an effective medium in which a yeast strain can be cultured. An effective medium may be an aqueous medium comprising assimilatable carbohydrate, nitrogen and phosphate sources, as well as appropriate salts, minerals, metals and other nutrients, such as vitamins and growth factors. The medium may comprise complex nutrients or may be a defined minimal medium. Yeast cells may be cultured in a variety of containers, including, but not limited to, bioreactors, test tubes, microtiter dishes, and petri plates. Culturing is carried out at a temperature, pH and oxygen content appropriate for the yeast strain. Such culturing conditions are well within the expertise of one of ordinary skill in the art (see, for example, Guthrie et al. (eds.), 1991, Methods in Enzymology, vol. 194, Academic Press, San Diego).

The level of antigen expressed in yeast cells and YCPs may be determined by any method for measuring antigen levels. Exemplary methods include ELISA, Western Blot, fluorimetric or FACS analysis using an antibody that specifically binds to the antigen.

For vaccines that induce immunity, the amount of antigen present in a YCP is preferably an amount that is sufficient for inducing immunity, e.g., at least about 1%, 2%, 3%, 4%, 5%, 6%, 7%, 8%, 9%, 10%, 15% or 20% of total protein. For vaccines that induce tolerance (e.g., for treating allergy), the amount of antigen present in a YCP is preferably an amount that is less than an amount that induces immunity, e.g., less than about 1%, less than about 0.5%, or less than about 0.1% of total protein. Amounts of antigen may also be expressed as % dry weight, % β-glucan or mannan, or on a pg per YCP basis. For example, since a yeast cell contains about 6 pg of total protein, an antigen present in 1 to 20% of total protein would correspond to about 0.06 to 0.12 pg/YCP.

Scaffolds

For retaining an antigen in a yeast cell while processing it into a YCP, an antigen is preferably present in a form that reduces its diffusion from the yeast cell as that cell is processed into a YCP. If an unmodified expressed antigen is naturally present in the yeast cytoplasm as a soluble protein, then the antigen is preferably modified so that it aggregates and/or becomes insoluble in the yeast cytoplasm. However, if an antigen is naturally present in the yeast cytoplasm in an aggregated form, it does not have to be further modified. “Aggregates” include both ordered multi-protein complexes, as in virus-like particles, and disordered complexes.

In one embodiment, a modification of an antigen comprises linking it to a scaffold or aggregation peptide or protein. One type of scaffold is a viral protein, e.g., a viral capsid, coat, envelope or core protein that self-assembles in the yeast cytoplasm to form virus-like particles (VLPs). Preferable viral proteins would have an N- or C-terminus exposed on the VLP surface so that large antigens could be attached without disrupting VLP assembly. An example of such a scaffold is hepatitis B surface antigen (HBsAg) (Yamaguchi et al. (1998) FEMS Microbiol. Lett. 165:363 and examples) or a portion thereof, which is used, e.g., in the commercial Engerix B vaccine. Nucleotide and amino acid sequences of an HBsAg from one Hepatitis B virus isolate are set forth in GenBank Accession Number AY040803 and AAK94662, respectively. Sequences from other Hepatitis B viral isolates may also be used. Portions of HsBAgs may also be used, e.g., a portion consisting essentially of about amino acids 100, 150 or 175 to about amino acid 400 of HsBAg may be used. An exemplary portion of an HsBAg has SEQ ID NO: 18. N-terminal antigen fusions to HbsAg (Wunderlich et al. (2000) Mol. Med. 6:238) have been used to elicit specific immune responses (Palucha et al. (2005) Prog. Nucleic Acid Res. Mol. Biol. 80:135).

A hepatitis B (HB) core protein may also be used. Nucleotide and amino acid sequences of an exemplary HB core protein are set forth in GenBank Accession numbers X85272 and CAA59535, respectively. The nucleotide sequence (161.718 of X85272) encoding this HB core protein is the following:
atggacatcgacccttataaagaatttggagcttctgcggagttactctcgtttttgccttctgacttctttccttccgtccgggatctactagacac agccaaagctttgtttcaggaagccttagagtctcctgagcattgttcgcctcaccatactgcactcaggcaagcaattctttgctggggggac ctaatgactctagctacctgggtgggtgctaatttggaagatccagcttctagagacctagtagtcaattatgtcaacactactgcgggcctaa agttcagacaactcttgtggtttcacatttcttgtctcacttttggaagagaaacagtgatagagtatttggtgtctttcggagtgtggattcgcact cctccaccttatagaccaccaaatgcccctatcttatcaacacttccggaaactactgttgttagataccgagaccgaggcaggtccactaga agaagaactccctcgcctcgcagacgaagatctcaatcgccgcgtcgcagaagatctcaatctcgggaatctcaatgttag (SEQ ID NO: 15) and the protein encoded thereby has the following amino acid sequence:
MDIDPYKEFGASAELLSFLPSDFFPSVRDLLDTAKALFQEALESPEHCSPHHTALRQAILC WGDLMTLATWVGANLEDPASRDLVVNYVNTTAGLKFRQLLWFHISCLTFGRETVIEYL VSFGVWIRTPPPYRPPNAPILSTLPETTVVRYRDRGRSTRRRTPSPRRRRSQSPRRRRSQS RESQC (SEQ ID NO: 16). Similar proteins from other isolates may also be used, as well as portions thereof, e.g., amino acids 1-149 (see Examples).

Another example is the mouse polyoma VP1 protein (Sasnauskas et al. (2002) Intervirology 45:308 and examples). VP1 of polyomaviruses from various species may be used, e.g., polyomaviruses from humans (JC polyomavirus and serotypes AS and SB of BK polyomavirus), rhesus monkeys (simian virus 40), hamsters (hamster polyomavirus), mice (murine polyomavirus) and birds (budgerigar fledgling disease virus). An exemplary polyoma VP1 protein has the amino acid sequence set forth as SEQ ID NO: 20. Fusions to VP 1 (Tegerstedt et al. (2005) Anticancer Res. 25:2601) have been used to elicit specific immune responses (Palucha et al. (2005) Prog. Nucleic Acid Res. Mol. Biol. 80:135).

Capsid, envelope, coat or core proteins from other viruses that are capable of aggregating in yeast cytoplasm may also be used. Amino acid and nucleotide sequences of VLP proteins may be found, e.g., in GenBank and the literature. For example, the amino acid and nucleotide sequences of the VP1 capsid protein of murine pneumotropic virus is provided in GenBank Accession number NP_{—}41234 and M55904, respectively. The amino acid and nucleotide sequences of the VP1 capsid protein of the BK polyomavirus is provided in GenBank Accession number NP_{—}041397 and V01108, respectively. Any fragment of a VLP that is capable of self-assembly may also be used.

Another type of scaffold is a small self-aggregating peptide, such as from yeast. Such peptides may be asparagine-glutamine (NQ)-rich and spontaneously aggregate into stacked cross-β sheet fibrils when over-expressed, e.g., from the fully induced GAL1p promoter (Edskes et al. (1999) PNAS 96: 1498 and Ripaud et al. (2003) EMBO J. 22:5251).

An exemplary small self-aggregating peptide that may be used as scaffold is a peptide of the N-terminus of the yeast Saccharomyces cerevisiae Ure2p enzyme (Edskes et al. (1999) PNAS 96:1498). The S. cerevisiae Ure2p protein has Gene ID: 855492 and its amino acid sequence is set forth in GenBank Accession No. NP_{—}014170 and is the following:

(SEQ ID NO:24)

MMNNNGNQVSNLSNALRQVNIGNRNSNTTTDQSNINFEFSTGVNNNNNNN
SSSNNNNVQNNNSGRNGSQNNDNENNIKNTLEQHRQQQQAFSDMSHVEYS
RITKFFQEQPLEGYTLFSHRSAFNGFKVAIVLSELGFHYNTIFLDFNLGE
HRAPEFVSVNPNARVPALIDHGMDNLSIWESGAILLHLVNKYYKETGNPL
LWSDDLADQSQINAWLFFQTSGHAPMIGQALHFRYFHSQKIASAVERYTD
EVRRVYGVVEMALAERREALVMELDTENAAAYSAGTTPMSQSRFFDYPVW
LVGDKLTIADLAFVPWNNVVDRIGINIKIEFPEVYKWTKHMMRRPAVIKA
LRGE.

The amino acid sequence of a S. cerevisiae Ure2p protein is also set forth in GenBank Accession No. AAM93174 and is encoded by the nucleotide sequence set forth in AF525181.

A peptide of Ure2p for use as a scaffold may correspond to about amino acids 1-65; 1-67, 1-70, 1-75, 1-76, 1-80, 1-85, 1-89, 1-90, 1-95 or 1-100 of Ure2p (SEQ ID NO: 24). An exemplary N-terminal portion of Ure2p is set forth as SEQ ID NO: 22. Longer portions may also be used as a scaffold. Linking such a peptide or protein to an antigen confers to the antigen the property of forming an insoluble and protease-resistant fibrillar form.

The Ure2p protein or portions thereof of other species of yeasts may also be used, provided they are capable of forming insoluble fibrils. For example, the N-terminus of full length Ure2p of S. paradoxus and S. uvarum form fibrils and may thus be used as a scaffold (Baudin-Baillieu et al. (2003) Mol. Biol. Of the Cell. 14: 3449). In particular, the N-terminus of S. paradoxus Ure2p produces more stable aggregates than that of S. cerevisiae and thus forms a suitable scaffold. Immel et al. In Vitro Analysis of SpUre2p, a Prion-related Protein, exemplifies the Relationship between Amyloid and Prion. J Biol. Chem. 2007 Mar. 6; 282(11):7912-20.

Any other protein or peptide capable of forming fibrils may be used. For example, several other yeast proteins form fibrils. For example, Sup35p, whose fibrillar form is referred to as [PSI], or portions thereof, from various species may be used. Other yeast fibril-forming proteins include Rnq1p and New1 from S. cereviseae (Santoso et al. (2000) Cell 100:277 and Sondheimer et al. (2000) Mol. Cell. 5:163). The full length proteins or fragments thereof may be used.

Generally, any protein, peptide or fragment thereof that spontaneously aggregates into fibrils in the yeast cytoplasm can potentially be used as a YCP antigen scaffold. Accordingly, other scaffolds that may be used include the following.

1. Natural fibril forming proteins of bacterial origin such as Curli from E. coli, specifically CsgA and CsgB proteins. (Chapman M R, Robinson L S, Pinkner J S, Roth R, Heuser J, Hammar M, Normark S, Hultgren S J Role of Escherichia coli curli operons in directing amyloid fiber formation. Science. 2002 Feb. 1; 295(5556):851-5).

2. Natural fibril forming proteins of fungal origin such as the HET-s protein of Podospora anserine, specifically residues 218-289 (Ritter, R. Riek, et al Nature 2005. 435: 844-8 and Balguerie A, Dos Reis S, Ritter C, Chaignepain S, Coulary-Salin B, Forge V, Bathany K, Lascu I, Schmitter J M, Riek R, Saupe S J. Domain organization and structure-function relationship of the HET-s prion protein of Podospora anserina. EMBO J. 2003 May 1; 22(9):2071-81). Alternatively, just the beta-sheet amyloid-forming peptide fragment of HetS may be used (Balbirnie M, Grothe R, Eisenberg D S. An amyloid-forming peptide from the yeast prion Sup35 reveals a dehydrated beta-sheet structure for amyloid. Proc Natl Acad Sci USA. 2001 Feb. 27; 98(5):2375-80; Eisenberg, D et al., abstract 009, Keystone meeting on Protein misfolding diseases, Breckenridge, Feb. 21-26, 2006).

3. A seven residue peptide, GQQNNYN (SEQ ID NO: 25), derived from the yeast Sup35 protein that self-assembles into fibers in vitro (Nelson, R. Sawaya, M. R., Balbirnie, M., Madsen, A. O., Riekel, C., Grothe, R., Eisenberg, 2005 Nature 435 773-8. Structure of the cross-beta spine of amyloid-like fibrils) or a synthetic peptide (SSTSAA) that behaves similarly (David Eisenberg, personal communication) or dimeric forms of these peptides linked by a short peptide sequence capable of forming an appropriate turn.

4. Natural fibril forming proteins of mammalian origin such as

a. the A-beta 1-42 fragment of the human APP (Luhrs, T., Ritter, C., Adrian, M., Riek-Loher, D., Bohrmann, B., Dobeli, H., Schubert, D., Riek, R. 2005 Proc Natl Acad Sci USA, 102: 17342-7. 3D structure of Alzheimer's amyloid-beta (1-42) fibrils) or just the 37-42 fragment thereof (Eisenberg, D et al., abstract 009, Keystone meeting on Protein misfolding diseases, Breckenridge, Feb. 21-26, 2006).

b. the luminal fragment of the Pme117 protein of human melanocytes (Fowler D M, Koulov A V, Alory-Jost C, Marks M S, Balch W E, Kelly J W. Functional Amyloid Formation within Mammalian Tissue. PLoS Biol. 2005 Nov. 29; 4(1):e6).

c. the 67 residue proIAPP precursor of the islet amyloid peptide (Amylin), or the 37 residue IAPP itself (Tatarek-Nossol, M., Yan, L. M., Schmauder, A., Tenidis, K., Westermark, G., Kapurniotu, A. 2005. Chem Biol 12: 797-809. Inhibition of hIAPP amyloid-fibril formation and apoptotic cell death by a designed hIAPP amyloid-core-containing hexapeptide; and Paulssen, J and Westermark, G. T, abstract 225, Keystone meeting on Protein misfolding diseases, Breckenridge, Feb. 21-26, 2006).

5. Synthetic poly asparagines such as N104 (Peters, T. and Huang, M. abstract 227, Keystone meeting on Protein misfolding diseases, Breckenridge, Feb. 21-26, 2006).

6. Additional virus-like particles, suitable as scaffolds, that are derived from S. cerevisiae:

a. the capsid (gag protein) of the L-A dsRNA virus, e.g., expressed from a cDNA (Caston J R, Trus B L, Booy F P, Wickner R B, Wall J S, Steven A C. J. Cell Biol. 1997 Sep. 8; 138(5):975-85. Structure of L-A virus: a specialized compartment for the transcription and replication of double-stranded RNA and Juan Carlos Ribas and Reed B. Wickner J Biol Chem, Vol. 273, Issue 15, 9306-9311, Apr. 10, 1998 The Gag Domain of the Gag-Pol Fusion Protein Directs Incorporation into the L-A Double-stranded RNA Viral Particles in Saccharomyces cerevisiae).

b. the capsid of the Ty retroposon. (Kingsman A J, Burns N R, Layton G T, Adams S E. Yeast retrotransposon particles as antigen delivery systems. Ann N Y Acad. Sci. 1995 May 31; 754:202-13) The yeast TY sequence is included in GenBank Accession No. SCTyl09.

7. Any synthetic peptide that is designed to adopt the dehydrated core structure characteristic of all amyloid fibrils (Balbirnie M, Grothe R, Eisenberg D S. An amyloid-forming peptide from the yeast prion Sup35 reveals a dehydrated beta-sheet structure for amyloid. Proc Natl Acad Sci U S A. 2001 Feb. 27; 98(5):2375-8)

In another embodiment, a scaffold comprises or consists of a peptide that can be aggregated by a change in the environment, such as the addition of a molecule. For example, the scaffold may be avidin or streptavidin and the presence of a molecular complex comprising several biotin molecules will result in an aggregation of the antigen. In another embodiment, a His tag, e.g., a hexahistidine (His₆), is used as a scaffold, wherein the presence of a metal, e.g., Nickel in the form of a molecular complex, allows the antigen-scaffold fusion proteins to aggregate. Methods for aggregating proteins may also use other affinity interactions, such as using polyelectrolytes and other reagents described in U.S. 20050281781.

A person of skill in the art would understand that functional homologs of any of the naturally-occurring scaffolds, e.g., proteins forming VLPs and prion-like proteins, and portions thereof may be used. Functional homologs include homologs having a certain similarity in amino acid or nucleotide sequence to the naturally-occurring protein and/or portions of the naturally-occurring proteins.

Exemplary homologs include those having an amino acid sequence that differs from that of a naturally-occurring scaffold protein by 1, 2, 3, 4, 5, 10 or more amino acid deletions, insertions of substitutions. Substitutions may be conserved amino acid substitutions. Homologs may also comprise or consist of an amino acid sequence that is at least about 60%, 70%, 80%, 90%, 95%, 98% or 99% identical to that of a naturally-occurring scaffold protein or a portion thereof. Other homologs include those that are encoded by a nucleic acid that comprises a nucleotide sequence that is at least about 60%, 70%, 80%, 90%, 95%, 98% or 99% identical to that of a naturally-occurring nucleic acid encoding a scaffold protein or a portion thereof. Yet other homologs include those that are encoded by a nucleic acid that hybridizes under low, medium or high stringency conditions to a nucleic acid encoding a naturally-occurring scaffold protein or a portion thereof. Hybridization conditions are further described elsewhere herein.

Functional homologs also include homologs in which the amino acid sequence of a naturally-occurring protein has been scrambled. It has been shown that the sequence of the Ure2p N-terminal peptide can be scrambled without affecting aggregation propensity, probably because it retained a high NQ content (Ross et al. (2005) PNAS 102:12825). Exemplary scrambled sequences of amino acids 1-89 of the S. cerevisiae Ure2p are as follows (Ross et al., supra):

Scrambled sequence 1 of the 89 N-terminal amino acids of S. cerevisiae Ure2:

(SEQ ID NO:1)

MVDGNQMNNNKSRRNSSQRGNSNQRVNNQNENNFNGLAQSSNNNNSITTT
FTNNNQINSQLNGINNNVNQTDQNVQNHGNSNENNSENL

Scrambled sequence 2 of the 89 N-terminal amino acids of S. cerevisiae Ure2:

(SEQ ID NO:2)

MQSHQAESNSSQNGDQNGTNNLQNNRSNGINNFGNNRNQNNLESQRVNNT
INNNKLNQFNGNNEVNNVQNQSSDNTNNNTMSIVTTRNS

Scrambled sequence 3 of the 89 N-terminal amino acids of S. cerevisiae Ure2:

(SEQ ID NO:3)

MNIRNQNQSTAVLNVNQQSNNGTSNSVNNLNFNNSGMQNHGRNFNQSTRN
NNTNEKGGNNILNSNDERINNQQNQENNNTVDNSQNNSS

Scrambled sequence 4 of the 89 N-terminal amino acids of S. cerevisiae Ure2:

(SEQ ID NO:4)

MMQRNGQQEGTNNNHSNINTQRNVFNNSANNNRNNNEGLNNNNSNFNNLV
SNNQQVNVSSNSNINNQDNNKSILSGTSNDTTENRGQQQ

Scrambled sequence 5 of the 89 N-terminal amino acids of S. cerevisiae Ure2:

(SEQ ID NO:5)

MNTNNSQGSFVDENQNRSIVKSRTVNMSQNNNTGNNNNAQLNNILNNTDS
GHVSNNENRLGRQNNEFNQNSSQTNNGNNQQQSNNNNNI

Thus, other scrambled sequences of proteins having fiber forming properties or portions thereof may be used.

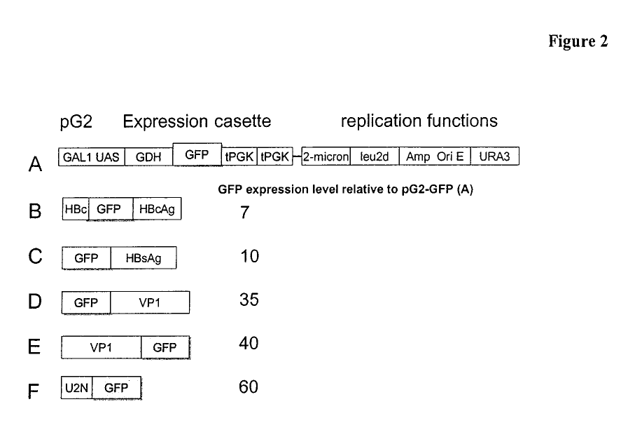

Antigens may be linked to scaffolds at the N-terminus, C-terminus or may be internal to the scaffold (see, e.g., FIG. 2, showing examples of green fluorescent protein (GFP) linked to HB core). In addition, scaffolds may be linked directly to the antigen, or through a linker, e.g., a flexible peptide linker. An example of a flexible peptide linker is a peptide consisting of about 5-30 amino acids; about 10-20 amino acids or about 10-15 amino acids, e.g., about 13 amino acids. A flexible peptide linker may comprise, consist essentially of, or consist of the amino acid sequence GGTSGGSTGLSSG (SEQ ID NO: 6); LDGTSGGSGSSS (SEQ ID NO: 7);

(SEQ ID NO:8)

GGTSGGSTGLESSG,

(SEQ ID NO:9)

GGSSGGSSGLDSS
or

(SEQ ID NO:23)

GGSSGGSS.

Also provided herein are nucleic acids encoding an antigen fused directly or indirectly to a scaffold and yeast cells comprising such.

Antigens and Methods of Treatment

Provided herein are methods for inducing an immune response to an antigen in a subject. In one embodiment, a method comprises administering to a subject, e.g., a subject in need thereof, a therapeutically effective amount of a YCP comprising an antigen, such as to induce a protective immune response in the subject to a microorganism comprising a similar antigen. Accordingly, YCPs comprising one or more antigens may be used to therapeutically or prophylactically to treat a subject who is or may become infected with an infectious agent or pathogen. An exemplary method comprises administering to a subject, such as a subject who is or is likely to become infected with a pathogen, a therapeutically effective amount of a YCP comprising an antigen from the pathogen or a functional homolog thereof.

A person (or subject) in need of treatment of prevention may be a person that has been exposed to or is likely to be exposed to or infected with a pathogenic microorganism or infectious agent, such as medical personnel that have been exposed to or likely to be exposed to bioterrorism. Other persons at risk of being exposed include, but are not limited to, military personnel, mail handlers, and governmental officials, as well as those with weakened immune systems, for example, the elderly, people on immunosuppressive drugs, subjects with cancer, and subjects infected with HIV.

Other subjects that may be treated include any animal susceptible to any disease from which a YCP can be designed to protect the animal. Exemplary animals that may be treated include vertebrates and arthropods, mammals, amphibians, bird, fish, insects, humans, primates, companion animals (i.e., pets) and agriculturally important animals (i.e., livestock), apes, cats, cattle, dogs, ferrets, birds, fowl, gorillas, horses, mice, monkeys, pigs, rabbits, rats and sheep.

YCPs may also be used for reducing the frequency of incidence of a disease that it transmitted by a non-human animal, e.g., plague, in a human population that is contiguous to an animal population reservoir. A method may comprise administering to the animal population a YCP described herein. For example, it may be desirable to immunize rats, which transmit the plague, with a YCP comprising Yersinia pestis LcrV.

An infectious agent can be any agent that can infect an organism, e.g., an animal, and cause or increase the risk of causing an undesirable effect on the organism, e.g., a disease. Exemplary infectious agents against which to protect organisms using YCPs include prokaryotic and eukaryotic microorganisms, such as bacteria, fungi (including yeast), protozoa (e.g., amebas, flagellates and sporozoa), helminths, ectoparasites, and viruses.

In one embodiment, YCPs deliver antigens of bioterrorism critical biological agents, such as National Institute of Allergy and Infectious Diseases (NIAID) priority pathogens. These include Category A agents, such as variola major (smallpox), Bacillus anthracis (anthrax), Yersinia pestis (plague), Clostridium botulinum toxin (botulism), Francisella tularensis (tularaemia), filoviruses (Ebola hemorrhagic fever, Marburg hemorrhagic fever), arenaviruses (Lassa (Lassa fever), Junin (Argentine hemorrhagic fever) and related viruses); Category B agents, such as Coxiella burnetti (Q fever), Brucella species (brucellosis), Burkholderia mallei (glanders), alphaviruses (Venezuelan encephalomyelitis, eastern & western equine encephalomyelitis), ricin toxin from Ricinus communis (castor beans), epsilon toxin of Clostridium perfringens; Staphylococcus enterotoxin B, Salmonella species, Shigella dysenteriae, Escherichia coli strain O157:H7, Vibrio cholerae, Cryptosporidium parvum; and Category C agents, such as nipah virus, hantaviruses, tickborne hemorrhagic fever viruses, tickborne encephalitis viruses, yellow fever, and multidrug-resistant tuberculosis.

Other exemplary prokaryotic and eukaryotic pathogens against which YCPs may protect a subject include bacteria, such as bacteria from the genus Aspergillus, Brugia, Candida, Chlamydia, Coccidia, Cryptococcus, Dirofilaria, Gonococcus, Histoplasma, Leishmania, Mycobacterium, Mycoplasma, Paramecium, Pertussis, Plasmodium, Pneumococcus, Pneumocystis, Rickettsia, Salmonella, Shigella, Staphylococcus, Streptococcus, Toxoplasma and Vibriocholerae. Exemplary species include Neisseria gonorrhea, Mycobacterium tuberculosis, Candida albicans, Candida tropicalis, Trichomonas vaginalis, Haemophilus vaginalis, Group B Streptococcus sp., Microplasma hominis, Hemophilus ducreyi, Granuloma inguinale, Lymphopathia venereum, Treponema pallidum, Brucella abortus. Brucella melitensis, Brucella suis, Brucella canis, Campylobacterfetus, Campylobacterfetus intestinalis, Leptospira pomona, Listeria monocytogenes, Brucella ovis, Chlamydia psittaci, Trichomonasfoetus, Toxoplasma gondii, Escherichia coli, Actinobacillus equuli, Salmonella abortus ovis, Salmonella abortus equi, Pseudomonas aeruginosa, Corynebacterium equi, Corynebacterium pyogenes, Actinobaccilus seminis, Mycoplasma bovigenitalium, Aspergillus fumigatus, Absidia ramosa, Trypanosoma equiperdum, Babesia caballi, Clostridium tetani, Clostridium botulinum, as well as other infectious agents that cause opportunistic infections in animals that are immunodeficient or otherwise immunosuppressed. Other infectious agents include harmful microorganisms found in brackish water, food contaminants and wounds.

Any protein or functional homolog thereof of a protein from a pathogen may be used as an antigen in a YCP provided that it is immunogenic and allows an immune response to be raised against the pathogen comprising the antigen. For example, an antigen that may be used for protection against anthrax is B. anthracis protective antigen (PA), which is a component of the toxin delivery system. The It has been shown that intranasally-delivered PA provides protective immunity against the anthrax toxin (Sloat et al. (2005) Pharm. Res., in press, and Sheff et al. (2004) Yeast 21:661). It has also been shown that vaccines made using the 63 kDa mature form of PA, purified from yeast expressing it in codon-optimized form, provide protection against anthrax spores in non-human primates (Hepler et al. (2005) Vaccine, in press). Thus, a YCP comprising PA or a functional homolog thereof linked to a scaffold may be used as a vaccine, e.g., an oral vaccine, against anthrax.

An antigen that may be used for protection against influenza is influenza hemagglutinin (HA) or a functional homolog thereof. For example, an antigen that may be used for to induce protection against the avian flu, such as that caused by H5N1 influenza, is influenza H5 hemagglutinin. Such a vaccine could be used as a vaccine in both humans and domestic fowl. An HA produced in yeast (Pichia) in a secreted form has been shown to be effective in protecting mice against a lethal influenza virus challenge (Saelens et al. (1999) Eur. J. Biochem. 260:166).

An antigen that may be used for protection against plague is the Yersinia pestis LcrV protein or a functional homolog thereof, which protein is a component of the type III secretion apparatus that is essential for virulence.

TABLE 1
Antigens that may be used

| | | | GenBank | | |
|---|---|---|---|---|---|
| | | | acc. | | |
| | | | for | | Conserved |
| | | | nucleotide | GenBank acc. | domains |
| Antigen | Pathogen | Gene ID | sequ. | for amino acid sequ. | (amino acids) |
| Protective | Bacillus anthracis str. | 2820165 | AE017336 | YP_016495 | 132-573 |
| antigen | Ames Ancestor | | | | |
| Protective | Bacillus anthracis str. | 1158723 | AE011190 | NP_652920 | 132-573 |
| antigen | A2012 | | | | |
| Hemagglutinin | Influenza A virus | 3655103 | L11133 | YP_308850; | |
| | (A/Korea/426/68(H2N2))] | | | Mature peptide | |
| | | | | YP_308873 HA1; | |
| | | | | Mature peptide | |
| | | | | YP_308874 HA2 | |
| Hemagglutinin | Influenza A virus (A/New | 3655151 | CY002064 | YP_308839; | 51-145 |
| | York/392/2004(H3N2)) | | | Mature peptide | |
| | | | | YP_308875 HA1; | |
| | | | | Mature peptide | |
| | | | | YP_308876 HA2 | |
| Hemagglutinin | Influenza A virus | 3654620 | AF144305 | YP_308669 | |
| | (A/Goose/Guangdong/1/96 | | | | |
| | (H5N1)) | | | | |
| Hemagglutinin | Influenza A virus | 1460996 | AJ404626 | NP_859037 | |
| | (A/Hong | | | | |
| | Kong/1073/99(H9N2)) | | | | |
| Hemagglutinin | Influenza B virus | 956538 | K00423 | NP_056660 | |
| hemagglutinin- | Influenza C virus | 3077359 | AB126194 | YP_089655 | |
| esterase | | | | | |
| precursor | | | | | |
| LcrV | Yersinia enterocolitica | 1239194 | AY150843 | NP_783665 | |
| | | 1449458 | AF336309 | NP_863514 | |
| | | 1239123 | AF102990 | NP_052392 | |
| LcrV | Yersinia pestis biovar | 2767532 | AE017043 | NP_995380 | |
| | Medievalis str. 91001 | | | | |
| LcrV | Yersinia pestis KIM | 1149310 | | NP_857946 | |
| | | 1149118 | | NP_857751 | |
| LcrV | Yersinia pestis CO92 | 1172676 | AL117189 | NP_395165 | |
| LcrV | Yersinia | 2952950 | BX936399 | YP_068466 | |
| | pseudotuberculosis IP | | | | |
| | 32953 | | | | |

Additional toxins produced by NIAID priority pathogens that are potential targets for this vaccine strategy include ricin, using as antigen a non-toxic ricin mutant selected in yeast (Allen et al. (2005) Yeast 22:1287); a C-terminal heavy chain fragment from botulinum neurotoxin serotype E (Dux et al. (2005) Protein Expr. Purif, in press); and the B subunit of the type II shiga toxin that is the dominant hemorrhagic toxin produced by most enterohemorrhagic strains of E. coli (Marcato et al. (2005) Infect. Immun. 73:6523).

Other toxins that YCPs may protect against, e.g., by including in the YCPs inactivated forms of toxins, such as anatoxin antigens, including toxoids (inactivated but antigenic toxins), and toxoid conjugates include: pertussis toxoid, Corynebacterium diphtheriae toxoid, tetanus toxoid, Haemophilus influenzae type b-tetanus toxoid conjugate, Clostridium botulinum D toxoid, Clostridium botulinum E toxoid, toxoid produced from Toxin A of Clostridium difficile, Vibrio cholerae toxoid, Clostridium perfringens Types C and D toxoid, Clostridium chauvoei toxoid, Clostridium novyi (Type B) toxoid, Clostridium septicum toxoid, recombinant HIV tat IIIB toxoid, Staphylococcus toxoid, Actinobacillus pleuropneumoniae Apx I toxoid, Actinobacillus pleuropneumoniae Apx II toxoid, Actinobacillus pleuropneumoniae Apx III toxoid, Actinobacillus pleuropneumoniae outer membrane protein (OMP) toxoid, Pseudomonas aeruginosa elastase toxoid, snake venom toxoid, Mannheimia haemolytica toxoid, Pasteurella multocida toxoid, Salmonella typhimurium toxoid, Pasteurella multocida toxoid, and Bordetella bronchiseptica toxoid. Recombinant methods of converting a toxin to a toxoid are known in the art (see, e.g., Fromen-Romano, C., et al., Transformation of a non-enzymatic toxin into a toxoid by genetic engineering, Protein Engineering vol. 10 no. 10 pp. 1213-1220, 1997).

Exemplary viruses from which to protect organisms using YCPs include Coxsackie viruses, cytomegaloviruses, Epstein-Barr viruses, flaviviruses, hepatitis viruses, herpes viruses, influenza viruses, measles viruses, mumps viruses, papilloma viruses, parainfluenza viruses, parvoviruses, rabies viruses, respiratory syncytial viruses, retroviruses, varicella viruses, adenoviruses, arena viruses, bunyaviruses, coronaviruses, hepadnaviruses, myxoviruses, oncogenic viruses, orthomyxoviruses, papovaviruses, paramyxoviruses, parvoviruses, picornaviruses, pox viruses, rabies viruses, reoviruses, rhabdoviruses, rubella viruses, togaviruses, equine herpes virus 1, equine arteritis virus, IBR-IBP virus, and BVD-MB virus. Other viruses include leukemia, lymphotrophic, sarcoma, lentiviruses and other immunodeficiency or tumor viruses. Exemplary lymphotrophic viruses against which a subject may be protected include T-lymphotrophic viruses, such as human T-cell lymphotrophic viruses (HTLVs, such as HTLV-I and HTLV-II), bovine leukemia viruses (BLVS) and feline leukemia viruses (FLVs). Particularly preferred lentiviruses include human (HIV), simian (SIV), feline (FIV) and canine (CIV) immunodeficiency viruses, with HIV-1 and HIV-2 being even more preferred.

Examples of viral antigens to be used in YCPs include env, gag, rev, tar, tat, nucleocapsid proteins and reverse transcriptase from immunodeficiency viruses (e.g., HIV, FIV); HBV surface antigen and core antigen; HCV antigens; influenza nucleocapsid proteins; parainfluenza nucleocapsid proteins; human papilloma type 16 E6 and E7 proteins; Epstein-Barr virus LMP-1, LMP-2 and EBNA-2; herpes LAA and glycoprotein D; as well as similar proteins from other viruses.

Accordingly, exemplary diseases from which subjects may be protected with YCPs comprising an antigen include anthrax, smallpox, plague, botulism, tularemia, and viral hemorrhagic fevers. Examples of fungal immunogenic and antigenic polypeptides include, but are not limited to, Absidia polypeptides, Acremonium polypeptides, Alternaria polypeptides, Aspergillus polypeptides, Basidiobolus polypeptides, Bipolaris polypeptides, Blastomyces polypeptides, Candida polypeptides, Coccidioides polypeptides, Conidiobolus polypeptides, Cryptococcus polypeptides, Curvalaria polypeptides, Epidermophyton polypeptides, Exophiala polypeptides, Geotrichum polypeptides, Histoplasma polypeptides, Madurella polypeptides, Malassezia polypeptides, Microsporum polypeptides, Moniliella polypeptides, Mortierella polypeptides, Mucor polypeptides, Paecilomyces polypeptides, Penicillium polypeptides, Phialemonium polypeptides, Phialophora polypeptides, Prototheca polypeptides, Pseudallescheria polypeptides, Pseudomicrodochium polypeptides, Pythium polypeptides, Rhinosporidium polypeptides, Rhizopus polypeptides, Scolecobasidium polypeptides, Sporothrix polypeptides, Stemphylium polypeptides, Trichophyton polypeptides, Trichosporon polypeptides, and Xylohypha polypeptides.

Examples of protozoan parasite immunogenic and antigenic polypeptides include, but are not limited to, Babesia polypeptides, Balantidium polypeptides, Besnoitia polypeptides, Cryptosporidium polypeptides, Eimeria polypeptides, Encephalitozoon polypeptides, Entamoeba polypeptides, Giardia polypeptides, Hammondia polypeptides, Hepatozoon polypeptides, Isospora polypeptides, Leishmania polypeptides, Microsporidia polypeptides, Neospora polypeptides, Nosema polypeptides, Pentatrichomonas polypeptides, Plasmodium polypeptides, e.g., P. falciparum circumsporozoite (PfCSP), sporozoite surface protein 2 (PfSSP2), carboxyl terminus of liver state antigen 1 (PfLSA1 c-term), and exported protein 1 (PfExp-1), Pneumocystis polypeptides, Sarcocystis polypeptides, Schistosoma polypeptides, Theileria polypeptides, Toxoplasma polypeptides, and Trypanosoma polypeptides.

Examples of helminth parasite immunogenic and antigenic polypeptides include, but are not limited to, Acanthocheilonema polypeptides, Aelurostrongylus polypeptides, Ancylostoma polypeptides, Angiostrongylus polypeptides, Ascaris polypeptides, Brugia polypeptides, Bunostomum polypeptides, Capillaria polypeptides, Chabertia polypeptides, Cooperia polypeptides, Crenosoma polypeptides, Dictyocaulus polypeptides, Dioctophyme polypeptides, Dipetalonema polypeptides, Diphyllobothrium polypeptides, Diplydium polypeptides, Dirofilaria polypeptides, Dracunculus polypeptides, Enterobius polypeptides, Filaroides polypeptides, Haemonchus polypeptides, Lagochilascaris polypeptides, Loa polypeptides, Mansonella polypeptides, Muellerius polypeptides, Nanophyetus polypeptides, Necator polypeptides, Nematodirus polypeptides, Oesophagostomum polypeptides, Onchocerca polypeptides, Opisthorchis polypeptides, Ostertagia polypeptides, Parafilaria polypeptides, Paragonimus polypeptides, Parascaris polypeptides, Physaloptera polypeptides, Protostrongylus polypeptides, Setaria polypeptides, Spirocerca polypeptides Spirometra polypeptides, Stephanofilaria polypeptides, Strongyloides polypeptides, Strongylus polypeptides, Thelazia polypeptides, Toxascaris polypeptides, Toxocara polypeptides, Trichinella polypeptides, Trichostrongylus polypeptides, Trichuris polypeptides, Uncinaria polypeptides, and Wuchereria polypeptides.

Examples of ectoparasite immunogenic and antigenic polypeptides include, but are not limited to, polypeptides (including protective antigens as well as allergens) from fleas; ticks, including hard ticks and soft ticks; flies, such as midges, mosquitos, sand flies, black flies, horse flies, horn flies, deer flies, tsetse flies, stable flies, myiasis-causing flies and biting gnats; ants; spiders, lice; mites; and true bugs, such as bed bugs and kissing bugs.

YCPs comprising an antigen may also be used in methods for conferring a broad based protective immune response against hyperproliferating cells that are characteristic of hyperproliferative diseases, as well as a method of treating individuals suffering from hyperproliferative diseases. As used herein, the term “hyperproliferative diseases” is meant to refer to those diseases and disorders characterized by hyperproliferation of cells. Examples of hyperproliferative diseases include all forms of cancer and psoriasis. In an illustrative embodiment, a method is for treating a subject, such as a subject having or likely to develop a hyperproliferative disease, and comprises administering to the subject a therapeutically effective amount of a hyperproliferating cell-associated protein or functional homolog thereof. The hyperproliferating cell-associated protein or functional homolog thereof preferably induces an immune response against a cell comprising the hyperproliferating cell-associated protein. As used herein, the term “hyperproliferative-associated protein” is meant to refer to proteins that are associated with a hyperproliferative disease.

In order for the hyperproliferative-associated protein to be an effective immunogenic target, it is preferred that the protein is produced exclusively and/or at higher levels in hyperproliferative cells relative to normal cells. The protein is also preferably expressed at the surface of cells. A hyperproliferative-associated protein may be the product of a mutation of a gene that encodes a protein. A mutated gene may encode a protein that is nearly identical to the normal protein except it has a slightly different amino acid sequence, which results in a different epitope not found on the normal protein. Such target proteins include those which are proteins encoded by oncogenes such as myb, myc, fyn, and the translocation genes bcr/abl, ras, src, P53, neu, trk and EGRF. Other target proteins include tumor-specific immunoglobulin variable regions (e.g., B cell lymphoma idiotypes), GM2, Tn, sTn, Thompson-Friedenreich antigen (TF), melanoma differentiation antigens, Globo H, Le(y), MUC1, MUC2, MUC3, MUC4, MUC5AC, MUC5B, MUC7, carcinoembryonic antigens, beta chain of human chorionic gonadotropin (hCG beta), HER2/neu, PSMA, EGFRvIII, KSA, prostate specific antigen (PSA), PSCA, GP100, MAGE 1, MAGE 2, TRP 1, TRP 2, tyrosinase, MART-1, PAP, carcninoembryonic antigen (CEA), BAGE, MAGE, RAGE, heatshock proteins (HSPs, e.g., gp96) and related proteins. In addition to oncogene products as target antigens, target proteins for anti-cancer treatments and protective regimens include variable regions of antibodies made by B cell lymphomas, and variable regions of T cell receptors of T cell lymphomas which, in some embodiments, are also used as target antigens for autoimmune diseases. Other tumor-associated proteins can be used as target proteins, such as proteins which are found at higher levels in tumor cells, including the protein recognized by monoclonal antibody 17-1A and folate binding proteins. Other target proteins may be found, e.g., at www.cancer.gov/newscenter/pressreleases/cancervaccines.

While YCPs may be used to immunize an individual against one or more of several forms of cancer, YCPs may also be useful to prophylactically immunize an individual who is predisposed to develop a particular cancer or who has had cancer and is therefore susceptible to a relapse. Developments in genetics and biotechnology, as well as epidemiology, allow for the determination of probability and risk assessment for the development of cancer in an individual. Using genetic screening and/or family health histories, it is possible to predict the probability that a particular individual has for developing any one of several types of cancer.

Similarly, those individuals who have already developed cancer and who have been treated to remove the cancer, or are otherwise in remission, are particularly susceptible to relapse and reoccurrence. As part of a treatment regimen, such individuals can be immunized against the cancer that they have been diagnosed as having had in order to combat such a recurrence. Thus, once it is known that individuals have had a type of cancer and are at risk of a relapse, they can be immunized in order to prepare their immune systems to combat any future appearance of the cancer.

Also provided herein are methods of treating individuals suffering from autoimmune diseases and disorders by conferring a broad based protective immune response against targets that are associated with autoimmunity, including cell receptors and cells which produce “self”-directed antibodies recognizing self-antigens.

Exemplary T cell mediated autoimmune diseases include rheumatoid arthritis (RA), multiple sclerosis (MS), Sjogren's syndrome, sarcoidosis, insulin dependent diabetes mellitus (IDDM), autoimmune thyroiditis, reactive arthritis, ankylosing spondylitis, scleroderma, polymyositis, dermatomyositis, psoriasis, vasculitis, Wegener's granulomatosis, Crohn's disease and ulcerative colitis. Each of these diseases is characterized by T cell receptors that bind to endogenous antigens and initiate the inflammatory cascade associated with autoimmune diseases. Vaccination against the variable region of the T cells would elicit an immune response including CTLs to eliminate those T cells.

In RA, several specific variable regions of T cell receptors (TCRs) which are involved in the disease have been characterized. These TCRs include Vβ-3, Vβ3-14, Vβ3-17 and Vβ-17 (see, e.g., Howell, M. D., et al., 1991 Proc. Natl. Acad. Sci. USA 88:10921-10925; Paliard, X., et al., 1991 Science 253:325-329; Williams, W. V., et al., 1992 J. Clin. Invest. 90:326-333). Thus, vaccination with a YCP that delivers at least one of these proteins or a functional homolog thereof is expected to elicit an immune response that will target T cells involved in RA.

In MS, several specific variable regions of TCRs which are involved in the disease have been characterized. These TCRs include Vβ-7 and Vα-10 (see, e.g., Wucherpfennig, K. W., et al., 1990 Science 248:1016-1019 and Oksenberg, J. R., et al., 1990 Nature 345:344-346). Thus, vaccination with a YCP that delivers at least one of these proteins or a functional homolog thereof is expected to elicit an immune response that will target T cells involved in MS.

In scleroderma, several specific variable regions of TCRs which are involved in the disease have been characterized. These TCRs include Vβ-6, Vβ3-8, Vβ-14 and Vβ-16, Vβ-3C, Vα-7, Vα-14, Vα-15, Vα-16, Vα-28 and Vα-12. Thus, vaccination with a YCP that delivers at least one of these proteins or a functional homolog thereof is expected to elicit an immune response that will target T cells involved in scleroderma.

In order to treat patients suffering from a T cell mediated autoimmune disease, particularly those for which the variable region of the TCR has yet to be characterized, a synovial biopsy can be performed. Samples of the T cells present can be taken and the variable region of those TCRs identified using standard techniques. Vaccines can be prepared using this information.

Exemplary B cell mediated autoimmune diseases against which YCPs comprising an antigen may protect a subject include Lupus (SLE), Grave's disease, myasthenia gravis, autoimmune hemolytic anemia, autoimmune thrombocytopenia, asthma, cryoglobulinemia, primary biliary sclerosis and pernicious anemia. Each of these diseases is characterized by antibodies which bind to endogenous antigens and initiate the inflammatory cascade associated with autoimmune diseases. Vaccination against the variable region of such antibodies would elicit an immune response including CTLs to eliminate those B cells that produce the antibody.

In order to treat patients suffering from a B cell mediated autoimmune disease, the variable region of the antibodies involved in the autoimmune activity may have to be identified. If this is the case, a biopsy can be performed and samples of the antibodies present at a site of inflammation can be taken. The variable region of those antibodies can be identified using standard techniques and vaccines can be prepared using this information.

In the case of SLE, one antigen is believed to be DNA. Thus, in patients to be immunized against SLE, their sera can be screened for anti-DNA antibodies and a vaccine can be prepared which includes the variable region of such anti-DNA antibodies found in the sera.

Common structural features among the variable regions of both TCRs and antibodies are well known. The DNA sequence encoding a particular TCR or antibody can generally be found following well known methods such as those described in Kabat, et al. 1987 Sequence of Proteins of Immunological Interest U.S. Department of Health and Human Services, Bethesda Md. In addition, a general method for cloning functional variable regions from antibodies can be found in Chaudhary, V. K., et al., 1990 Proc. Natl. Acad. Sci. USA 87:1066.

YCPs may also be used for treating or preventing Alzheimer's disease. For examples, YCPs may comprise a fragment of the human APP protein, e.g., the 1-15 fragment of the A-beta 1-42 peptide derived from the human APP protein, containing the major B-cell epitopes but lacking A-beta-specific T-cell epitopes, coupled to a suitable promiscuous T-cell epitope.

YCPs comprising an antigen may also be used to tolerize a subject to a particular antigen. Such methods may be used for treating allergies and related illnesses or conditions, such as excema and asthma. In an exemplary embodiment, a method for treating a subject suffering from allergies or likely to suffer from allergies, comprises administering to the subject a therapeutically effective amount of an antigen that causes the allergy (e.g., an allergen), such that tolerance is induced in the subject. Tolerance may be induced by administration of amounts of antigen that are lower than those needed for inducing an immune response against the antigen.

YCPs may also be used as delivery vehicle of proteins against which no immune reaction is desired. It may be desirable in certain embodiments to suppress an active immune response against the proteins to prevent the immune system from reacting them. Exemplary antigens that may be delivered to a subject with YCPs include mammalian proteins, such as, e.g., growth hormone (GH), including human growth hormone, bovine growth hormone, and other members of the GH supergene family; growth hormone releasing factor; parathyroid hormone; thyroid stimulating hormone; lipoproteins; alpha-1-antitrypsin; insulin A-chain; insulin B-chain; proinsulin; follicle stimulating hormone; calcitonin; luteinizing hormone; glucagon; clotting factors such as factor VIIIC, factor IX tissue factor, and von Willebrands factor; anti-clotting factors such as Protein C; atrial natriuretic factor; lung surfactant; a plasminogen activator, such as urokinase or tissue-type plasminogen activator (t-PA); bombazine; thrombin; alpha tumor necrosis factor, beta tumor necrosis factor; enkephalinase; RANTES (regulated on activation normally T-cell expressed and secreted); human macrophage inflammatory protein (MIP-1-alpha); serum albumin such as human serum albumin; mullerian-inhibiting substance; relaxin A-chain; relaxin B-chain; prorelaxin; mouse gonadotropin-associated peptide; DNase; inhibin; activin; vascular endothelial growth factor (VEGF); receptors for hormones or growth factors; an integrin; protein A or D; rheumatoid factors; a neurotrophic factor such as bone-derived neurotrophic factor (BDNF), neurotrophin-3, -4, -5, or -6 (NT-3, NT-4, NT-5, or NT-6), or a nerve growth factor such as NGF-beta; platelet-derived growth factor (PDGF); fibroblast growth factor such as aFGF and bFGF; epidermal growth factor (EGF); transforming growth factor (TGF) such as TGF-alpha and TGF-beta, including TGF-beta1, TGF-beta2, TGF-beta3, TGF-beta4, or TGF-beta5; insulin-like growth factor-I and -II (IGF-I and IGF-II); des(1-3)-IGF-I (brain IGF-D; insulin-like growth factor binding proteins; CD proteins such as CD3, CD4, CD8, CD19 and CD20; osteoinductive factors; immunotoxins; a bone morphogenetic protein (BMP); T-cell receptors; surface membrane proteins; decay accelerating factor (DAF); a viral antigen such as, for example, a portion of the AIDS envelope; transport proteins; homing receptors; addressins; regulatory proteins; immunoadhesins; antibodies; and biologically active fragments or variants of any of the above-listed polypeptides.

The members of the GH supergene family include growth hormone, prolactin, placental lactogen, erythropoietin, thrombopoietin, interleukin-2, interleukin-3, interleukin-4, interleukin-5, interleukin-6, interleukin-7, interleukin-9, interleukin-10, interleukin-11, interleukin-12 (p35 subunit), interleukin-13, interleukin-15, oncostatin M, ciliary neurotrophic factor, leukemia inhibitory factor, alpha interferon, beta interferon, gamma interferon, omega interferon, tau interferon, granulocyte-colony stimulating factor, granulocyte-macrophage colony stimulating factor, macrophage colony stimulating factor, cardiotrophin-1 and other proteins identified and classified as members of the family.

In other embodiments, a YCP is used for delivering an antibody. The antibody may, e.g., bind to any of the above-mentioned molecules. Exemplary molecular targets for antibodies encompassed by the present invention include CD proteins such as CD3, CD4, CD8, CD19, CD20 and CD34; members of the HER receptor family such as the EGF receptor, HER2, HER3 or HER4 receptor; cell adhesion molecules such as LFA-1, Mol, p150,95, VLA-4, ICAM-1, VCAM and alphav/beta3 integrin including either alpha or beta subunits thereof (e.g. anti-CD11a, anti-CD18 or anti-CD11b antibodies); growth factors such as VEGF; IgE; blood group antigens; flk2/flt3 receptor; obesity (OB) receptor; protein C, etc.

In other embodiments, YCPs may be used to deliver enzymes, such as ribonuclease, neuramidinase, trypsin, glycogen phosphorylase, sperm lactic dehydrogenase, sperm hyaluronidase, adenosinetriphosphatase-, alkaline phosphatase, alkaline phosphatase esterase, amino peptidase, trypsin chymotrypsin, amylase, muramidase, acrosomal proteinase, diesterase, glutamic acid dehydrogenase, succinic acid dehydrogenase, beta-glycophosphatase, lipase, ATP-ase alpha-peptate gamma-glutamylotranspeptidase, sterol-3-beta-ol-dehydrogenase, DPN-di-aprorase.

A YCP may comprise more than one antigen, e.g., at least 2, 3, 4, 5 or more antigens. In one embodiment, one or more antigens are from the same target, e.g., pathogen, hyperproliferating cell or autoimmune cell. For example, two or more proteins or functional homologs from a bacterial pathogen may be used. In another embodiment, a YCP comprises one or more proteins or functional homologs from one target and one or more proteins or functional homologs from 2 or more different targets. Accordingly, YCPs may be designed to protect an animal from more than one disease, e.g., from infection by at least 2, 3, 4 or 5 pathogens, or against 2, 3, 4, or 5 hyperproliferative or autoimmune diseases or a combination thereof. For example, it takes 7 HPVs to cover 90% of the cervical cancer inducers (Science, 2005 5722: 618).

Alternatively, at least 2, 3, 4, 5 or more different YCPs may be administered to a subject. These may comprise antigens from the same and/or different targets. In yet another embodiment, a YCP comprises one antigen comprising antigenic sequences from at least 2, 3, 4, 5 or more different antigens that is expessed as a scaffolded polyprotein.

YCP Formulations and Vaccines

YCPs may be prepared and/or stored as a liquid composition or in a dried form (e.g., lyophilized or spray-dried) form. When in a liquid composition, YCPs may be frozen at −20° C. or lower temperatures in a composition, e.g., PBS.

In one embodiment, YCPs are administered orally to a subject. YCPs may be mixed with a pharmaceutically acceptable excipient, such as an isotonic buffer that is tolerated by a subject. Examples of such excipients include water, saline, Ringer's solution, dextrose solution, Hank's solution, and other aqueous physiologically balanced salt solutions. Nonaqueous vehicles, such as fixed oils, sesame oil, ethyl oleate, or triglycerides may also be used. Other useful formulations include suspensions containing viscosity enhancing agents, such as sodium carboxymethylcellulose, sorbitol, or dextran. Excipients can also contain minor amounts of additives, such as substances that enhance isotonicity and chemical stability. Examples of buffers include phosphate buffer, bicarbonate buffer and Tris buffer, while examples of preservatives include thimerosal, m- or o-cresol, formalin and benzyl alcohol.

Other liquid dosage forms for oral administration include pharmaceutically acceptable emulsions, solutions, suspensions, syrups, and elixirs. In addition to the YCPs, the liquid dosage form may contain inert diluents commonly used in the art, such as water or other solvents, isotonic saline, solubilizing agents and emulsifiers, as for example, ethyl alcohol, isopropyl alcohol, ethyl carbonate, ethyl acetate, benzyl alcohol, benzyl benzoate, propylene glycol, 1,3-butylene glycol, dimethylformamide, oils, in particular, almond oil, arachis oil, coconut oil, cottonseed oil, groundnut oil, corn germ oil, olive oil, castor oil, sesame seed oil, MIGLYOL™ glycerol, fractionated vegetable oils, mineral oils such as liquid paraffin, tetrahydrofurfuryl alcohol, polyethylene glycols, fatty acid esters of sorbitan, or mixtures of these substances, and the like. Besides such inert diluents, the composition can also include adjuvants, wetting agents, emulsifying and suspending agents, demulcents, preservatives, buffers, salts, sweetening, flavoring, coloring and perfuming agents. Suspensions, in addition to the active compound, may contain suspending agents, as for example, ethoxylated isostearyl alcohols, polyoxyethylene sorbitol or sorbitan esters, microcrystalline cellulose, hydrogenated edible fats, sodium alginate, polyvinylpyrrdidone, gum tragacanth, gum acacia, agar-agar, and cellulose derivatives such as sodium carboxymethylcellulose, methylcellulose, hydroxypropylmethylcellulose, aluminum metahydroxide, bentonite, or mixtures of these substances, and the like.

Formulations of a pharmaceutical composition of the invention that are suitable for oral administration can be prepared, packaged, and sold either in liquid form or in the form of a dry product intended for reconstitution with water or another suitable vehicle prior to use. In a non-liquid formulation, the excipient can comprise, for example, dextrose, human serum albumin, and/or preservatives to which sterile water or saline can be added prior to administration.

Known dispersing or wetting agents include naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively). Known emulsifying agents include lecithin and acacia. Known preservatives include methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Solid dosage forms for oral administration include capsules, tablets, powders, and granules. In such solid dosage forms, YCPs are optionally admixed with at least one inert customary excipient (or carrier) such as sodium citrate or dicalcium phosphate or (a) fillers or extenders, as for example, starches, lactose, sucrose, mannitol, or silicic acid; (b) binders, as for example, carboxymethylcellulose, alginates, gelatin, polyvinylpyrrolidone, sucrose, or acacia; (c) humectants, as for example, glycerol; (d) disintegrating agents, as for example, agar-agar, calcium carbonate, potato or tapioca starch, alginic acid, certain complex silicates, or sodium carbonate; (e) solution retarders, as for example, paraffin; (f) absorption accelerators, as for example, quaternary ammonium compounds; (g) wetting agents, as for example, cetyl alcohol or glycerol monostearate; (h) adsorbents, as for example, kaolin or bentonite; and/or (i) lubricants, as for example, talc, calcium stearate, magnesium stearate, solid polyethylene glycols, sodium lauryl sulfate, or mixtures thereof. In the case of capsules and tablets, the dosage forms may also comprise buffering agents.

A tablet comprising YCPs can, for example, be made by compressing or molding the YCPs, optionally with one or more additional ingredients. Compressed tablets can be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets can be made by molding, in a suitable device, a mixture of the YCPs, a pharmaceutically acceptable carrier, and at least sufficient liquid to moisten the mixture. Pharmaceutically acceptable excipients used in the manufacture of tablets include inert diluents, granulating and disintegrating agents, binding agents, and lubricating agents. Known dispersing agents include potato starch and sodium starch glycolate. Known surface active agents include sodium lauryl sulfate. Known diluents include calcium carbonate, sodium carbonate, lactose, microcrystalline cellulose, calcium phosphate, calcium hydrogen phosphate, and sodium phosphate. Known granulating and disintegrating agents include corn starch and alginic acid. Known binding agents include gelatin, acacia, pre-gelatinized maize starch, polyvinylpyrrolidone, and hydroxypropyl methylcellulose. Known lubricating agents include magnesium stearate, stearic acid, silica, and talc.

Solid compositions may also be used as fillers in soft or hard filled gelatin capsules using such excipients as lactose or milk sugar, as well as high molecular weight polyethylene glycols, and the like. Hard capsules comprising YCPs can be made using a physiologically degradable composition, such as gelatin. Such hard capsules comprise the YCPs, and can further comprise additional ingredients including, for example, an inert solid diluent such as calcium carbonate, calcium phosphate, or kaolin. Soft gelatin capsules comprising the YCPs can be made using a physiologically degradable composition, such as gelatin. Such soft capsules comprise the YCPs, which can be mixed with water or an oil medium such as peanut oil, liquid paraffin, or olive oil.

Solid dosage forms such as tablets, dragees, capsules, and granules can be prepared with coatings or shells, such as enteric coatings and others well known in the art. For example, an enteric-resistant coat, e.g., cellulose acetate phthalate (Lavelle (2006) Methods 38:84), which will ensure resistance to stomach acid and efficient delivery to the lower GI tract can be used. Solid dosage forms may also contain opacifying agents. Examples of embedding compositions that can be used are polymeric substances and waxes. The YCPs can also be in micro-encapsulated form, if appropriate, with one or more of the above-mentioned excipients.

YCPs can also be in a form that allows them to be released in a delayed manner. Delayed disintegration of YCPs in the gastrointestinal tract of a human provides sustained release and absorption of the YCPs, e.g. in the region of the Peyer's patches in the small intestine. By way of example, a material such as glyceryl monostearate or glyceryl distearate can be used to coat tablets. Other enteric coated systems, e.g., for delivery to the gastrointestinal system, including the colon, may be based on, e.g., methacrylate copolymers such as poly(methacrylic acid, methyl methacrylate), which are only soluble at pH 6 and above, so that the polymer only begins to dissolve on entry into the small intestine. The site where such polymer formulations disintegrate is dependent on the rate of intestinal transit and the amount of polymer present. For example, a relatively thick polymer coating is used for delivery to the proximal colon (Hardy et al., 1987 Aliment. Pharmacol. Therap. 1:273-280). Polymers capable of providing site-specific colonic delivery can also be used, wherein the polymer relies on the bacterial flora of the large bowel to provide enzymatic degradation of the polymer coat and hence release of the drug. For example, azopolymers (U.S. Pat. No. 4,663,308), glycosides (Friend et al., 1984, J. Med. Chem. 27:261-268) and a variety of naturally available and modified polysaccharides (see PCT application PCT/GB89/00581) can be used in such formulations. Further by way of example, tablets can be coated using methods described in U.S. Pat. Nos. 4,256,108; 4,160,452; and 4,265,874 to form osmotically-controlled release tablets. Tablets can further comprise a sweetening agent, a flavoring agent, a coloring agent, a preservative, or some combination of these in order to provide pharmaceutically elegant and palatable preparation.

Pulsed release technology such as that described in U.S. Pat. No. 4,777,049 can also be used to administer the YCPs to a specific location within the gastrointestinal tract. Such systems permit delivery at a predetermined time and can be used to deliver the YCPs, optionally together with other additives that my alter the local microenvironment to promote stability and uptake, directly without relying on external conditions other than the presence of water to provide in vivo release.

In other embodiments, YCPs can be prepared as nutraceuticals, i.e., in the form of, or added to, a food (e.g., a processed item intended for direct consumption), drink or a foodstuff (e.g., an edible ingredient intended for incorporation into a food prior to ingestion). Examples of suitable foods include candies such as lollipops, baked goods such as crackers, breads, cookies, and snack cakes, whole, pureed, or mashed fruits and vegetables, beverages, and processed meat products. Examples of suitable foodstuffs include milled grains and sugars, spices and other seasonings, and syrups. For agricultural use, this would include standard feed pellets as well as drinking water.

A pharmaceutical composition comprising YCPs can be prepared, packaged, or sold in a formulation suitable for pulmonary administration via the buccal cavity. Such compositions are conveniently in the form of dry powders for administration using a device comprising a dry powder reservoir to which a stream of propellant can be directed to disperse the powder or using a self-propelling solvent/powder-dispensing container such as a device comprising the YCPs suspended in a low-boiling propellant in a sealed container. Dry powder compositions may include a solid fine powder diluent such as sugar and are conveniently provided in a unit dose form. Low boiling propellants generally include liquid propellants having a boiling point below 65° F. at atmospheric pressure. Generally the propellant can constitute 50 to 99.9% (w/w) of the composition, and the active ingredient can constitute 0.1 to 20% (w/w) of the composition. The propellant can further comprise additional ingredients such as a liquid non-ionic or solid anionic surfactant or a solid diluent (preferably having a particle size of the same order as particles comprising the YCPs).

Pharmaceutical compositions comprising YCPs formulated for pulmonary delivery can also provide the YCPs in the form of droplets of a suspension. Such formulations can be prepared, packaged, or sold as aqueous or dilute alcoholic suspensions, optionally sterile, comprising the particulate delivery system, and can conveniently be administered using any nebulization or atomization device. Such formulations can further comprise one or more additional ingredients including a flavoring agent such as saccharin sodium, a volatile oil, a buffering agent, a surface active agent, or a preservative such as methylhydroxybenzoate.

The formulations described herein as being useful for pulmonary delivery are also useful for intranasal delivery of a pharmaceutical composition comprising YCPs. Another formulation suitable for intranasal administration is a coarse powder comprising YCPs. Such a formulation is administered in the manner in which snuff is taken i.e. by rapid inhalation through the nasal passage from a container of the powder held close to the nares.

Since oral vaccines preferentially elicit a mucosal immune response in both gut and lungs (Ogra et al. (2001) Clin. Microbil. Rev. 14:430 and Shin et al. (2005) FEMS Immunol. Med. Microbiol. 43:155), they may be particularly effective against aerosolized biowarfare pathogens, potentially including influenza (Madjid et al. (2003) J. R. Soc. Med. 96:345). In addition, YCP's should be sufficiently inexpensive for use as an edible vaccine in poultry against H5N1 avian influenza, thereby also protecting human populations by immunizing the major carrier.

Compositions for rectal or vaginal administration can be prepared by mixing YCPs with suitable non-irritating excipients or carriers such as cocoa butter, polyethylene glycol or a suppository wax, which are solid at ordinary room temperature, but liquid at body temperature, and therefore, melt in the rectum or vaginal cavity and release the YCPs. Such a composition can be in the form of, for example, a suppository, a retention enema preparation, and a solution for rectal or colonic irrigation. Suppository formulations can further comprise various additional ingredients including antioxidants and preservatives. Retention enema preparations or solutions for rectal or colonic irrigation can be made by combining the YCPs with a pharmaceutically acceptable liquid carrier. As is known in the art, enema preparations can be administered using, and can be packaged within, a delivery device adapted to the rectal anatomy of a human. Enema preparations can further comprise various additional ingredients including antioxidants and preservatives.

YCPs may also be administered parentarally. Parenteral administration of a pharmaceutical composition includes any route of administration characterized by physical breaching of a tissue of a human and administration of the pharmaceutical composition through the breach in the tissue. Parenteral administration thus includes administration of a pharmaceutical composition by injection of the composition, by application of the composition through a surgical incision, by application of the composition through a tissue-penetrating non-surgical wound, and the like. In particular, parenteral administration includes subcutaneous, intraperitoneal, intravenous, intraarterial, intramuscular, or intrasternal injection and intravenous, intraarterial, or kidney dialytic infusion techniques.

Compositions suitable for parenteral injection comprise YCPs combined with a pharmaceutically acceptable carrier, such as physiologically acceptable sterile aqueous or nonaqueous solutions, dispersions, suspensions, or emulsions, or may comprise sterile powders for reconstitution into sterile injectable solutions or dispersions. Examples of suitable aqueous and nonaqueous carriers, diluents, solvents, or vehicles include water, isotonic saline, ethanol, polyols (propylene glycol, polyethylene glycol glycerol, and the like), suitable mixtures thereof, triglycerides, including vegetable oils such as olive oil, or injectable organic esters such as ethyl oleate. Proper fluidity can be maintained, for example, by the use of a coating such as lecithin, by the maintenance of the required particle size in the case of dispersions, and/or by the use of surfactants. Such formulations can be prepared, packaged, or sold in a form suitable for bolus administration or for continuous administration. Injectable formulations can be prepared, packaged, or sold in unit dosage form, such as in ampules, in multi-dose containers containing a preservative, or in single-use devices for auto-injection or injection by a medical practitioner.

Formulations for parenteral administration include suspensions, solutions, emulsions in oily or aqueous vehicles, pastes, and implantable sustained-release or biodegradable formulations. Such formulations can further comprise one or more additional ingredients including suspending, stabilizing, or dispersing agents. In one embodiment of a formulation for parenteral administration, YCPs are provided in dry (i.e. powder or granular) form for reconstitution with a suitable vehicle (e.g. sterile pyrogen-free water) prior to parenteral administration of the reconstituted composition. The pharmaceutical compositions can be prepared, packaged, or sold in the form of a sterile injectable aqueous or oily suspension or solution. This suspension or solution can be formulated according to the known art, and can comprise, in addition to the YCPs, additional ingredients such as the dispersing agents, wetting agents, or suspending agents described herein. Such sterile injectable formulations can be prepared using a non-toxic parenterally-acceptable diluent or solvent, such as water or 1,3-butanediol, for example. Other acceptable diluents and solvents include Ringer's solution, isotonic sodium chloride solution, and fixed oils such as synthetic mono- or di-glycerides. Other parentally-administrable formulations which are useful include those which comprise the YCPs in microcrystalline form, in a liposomal preparation, or as a component of a biodegradable polymer system. Compositions for sustained release or implantation can comprise pharmaceutically acceptable polymeric or hydrophobic materials such as an emulsion, an ion exchange resin, a sparingly soluble polymer, or a sparingly soluble salt.

These compositions may also contain adjuvants such as preserving, wetting, emulsifying, and/or dispersing agents. Prevention of microorganism contamination of the compositions can be accomplished by the addition of various antibacterial and antifungal agents, for example, parabens, chlorobutanol, phenol, sorbic acid, and the like. It may also be desirable to include isotonic agents, for example, sugars, sodium chloride, and the like. Prolonged absorption of injectable pharmaceutical compositions can be brought about by the use of agents capable of delaying absorption, for example, aluminum monostearate and/or gelatin.

Other methods of administering YCPs include sublingual, topical, or transmucosal administration, or through whole body spray (see, e.g., WO 00/04920).

The compositions described herein are preferably given to an individual in a “prophylactically effective amount” or a “therapeutically effective amount,” this being sufficient to show benefit to the individual. The actual amount administered, and rate and time-course of administration, will depend, e.g., on the nature and severity of what is being treated or prevented. Prescription of treatment, e.g. decisions on dosage etc, is within the responsibility of general practitioners and other medical doctors.

An effective dose of a vaccine may provide immunity to a pathogen, e.g., a virulent pathogen, by at least about 2, 3, 4, or 5 or more orders of magnitude more than the level of immunity in a non-immunized subject. An effective or single dose may comprise, e.g., about 10³ to about 10¹³, 10⁴ to about 10⁸, 10⁵ to about 5×10⁷ or about 10⁸ to about 10¹² YCPs per kg body weight of the subject. A dose may comprise about 1 to 500 μg, about 500-1,000 μg, about 1 mg-500 mg, about 500 mg to 1,000 mg, or about 1 to 10 g of antigen.

Multiple dosages may be used as needed to provide the desired level of protection or treatment. For example, one or more boosters may be needed over time to maintain protection of a eukaryote. Boosters may be given, e.g., every 5-20, 5-10 days, every week, every two weeks, every three weeks, every month or every few months. Boosters may be administered a few times, e.g., 2, 3, 4, 5, 7, 9, 10 or more times. Boosters may also be given one or more months or years after the first administration.

Administration of YCPs may be preceded and/or followed by fasting. Fasting before oral dosing is predicted to increase YCP survival in the GI tract, while fasting post-dose is predicted to reduce competition by food particles for M-cell and DC uptake. Fasting may be for about 30 minutes, 1 hour, 2 hours, 3 hours, or 5 hours or more.

The level of protection provided to a subject after immunization may be determined by methods known in the art, such as by determining the level of antibodies and/or T cells (such as cytotoxic T lymphocytes (CTL)) specific for antigens from the microorganisms, produced in response to the immunization. The presence of specific CTLs can be detected using standard assays such as an assay for Cr⁵¹ release or for the secretion of IFN-γ. The presence of specific antibodies can be detected by assays such as ELISA using the antigens which are immobilized on a culture plate, or a standard proliferation assay for T-helper cells. Mucosal sIgA responses may also be determined.

Adjuvants may be added to enhance the antigenicity of YCPs if desired, but are generally not required to induce an effective immune response, since components of the YCPs generally serve as adjuvants. However, it may be desirable to enhance the Th2 response by decreasing the mannan content of YCP's or to enhance the Th1 response by increased mannan content and/or by the addition of known general enhancers of such responses such as CpG-rich DNA of bacterial origin. It is also possible to load into YCP-scaffolded antigen preparations other adjuvants, ranging from the aforementioned CPG, endotoxin, cholera toxins, and other adjuvants known in the art (see, e.g., U.S. patent publication No. 20050281781). Adjuvants also include mutants of the E. coli heat-labile toxin, e.g., mutant R192G (Maier M, Seabrook T J, Lernere C A. Vaccine. 2005 Oct. 25; 23(44):5149-59). Modulation of the humoral and cellular immune response by the adjuvants monophosphoryl lipid A (MPL), cholera toxin B subunit (CTB) and E. coli enterotoxin LT(R192G) have recently been described as enhancing intra-nasal presentation of Abeta peptides (Lernere C A, Keystone meeting 2-06). It is also possible to load into YCPs cytokines/growth factors, etc and/or gene encoding these factors to enhance or direct the immune response (GM-CSF, IL-4, IL-10, IL-12, gamma interferon, etc.) Other agents that may be administered to a subject that is being treated with YCPs described herein include anti-infectious agents, e.g., anti-fungal compounds, anti-viral compounds, and antibiotics. Antibiotics include, but are not limited to, amoxicillin, clarithromycin, cefuroxime, cephalexin ciprofloxacin, doxycycline, metronidazole, terbinafine, levofloxacin, nitrofurantoin, tetracycline, and azithromycin. Anti-fungal compounds, include, but are not limited to, clotrimazole, butenafine, butoconazole, ciclopirox, clioquinol, clioquinol, clotrimazole, econazole, fluconazole, flucytosine, griseofulvin, haloprogin, itraconazole, ketoconazole, miconazole, naftifine, nystatin, oxiconazole, sulconazole, terbinafine, terconazole, fluconazole, and tolnaftate. Anti-viral compounds, include, but are not limited to, zidovudine, didanosine, zalcitabine, stavudine, lamivudine, abacavir, tenofovir, nevirapine, delavirdine, efavirenz, saquinavir, ritonavir, indinavir, nelfinavir, saquinavir, amprenavir, and lopinavir. Anti-infectious agents also include hyper-immune globulin. Hyper-immune globulin is gamma globulin isolated from a donor, or from a pool of donors, that has been immunized with a substance of interest. Specifically, hyper-immune globulin is antibody purified from a donor who was repeatedly vaccinated against a pathogen. Another component that may be added to a YCP composition is CpG-rich bacterial DNA, which further stimulate THI responses.

When YCPs and an agent are administered to a recipient (e.g., a eukaryote) administration of the YCPs and the agent may be done simultaneously or sequentially.

Also provided herein are kits. A kit may comprise one or more doses of YCPs comprising one or more antigens and optionally a device for administration or delivery of the YCPs. By way of example, a delivery device can be a squeezable spray bottle, a metered-dose spray bottle, an aerosol spray device, an atomizer, a dry powder delivery device, a self-propelling solvent/powder-dispensing device, a syringe, a needle, a tampon, or a dosage-measuring container. When the YCPs are not in a form ready for administration, e.g., they are in a lyophilized form, a kit may also comprise a buffer, e.g., PBS, for reconstituting a solution ready for administration. The kit can further comprise an instructional material.

The present description is further illustrated by the following examples, which should not be construed as limiting in any way. The contents of all cited references (including literature references, issued patents, published patent applications and GenBank Accession numbers as cited throughout this application) are hereby expressly incorporated by reference. When definitions of terms in documents that are incorporated by reference herein conflict with those used herein, the definitions used herein govern.

### EXAMPLES

### Example 1

### Use of Green Fluorescent Protein (GFP) a Model Soluble Protein Antigen to Optimize Antigen Expression in Yeast

Although other yeast species are better suited for high-level expression of foreign proteins, and all share the β-1.3 glucan that is the signature component of fungal cell walls, Baker's yeast was chosen as host for oral vaccine production since it should have minimal associated safety issues. Although IgE responsive to yeast are found in about 1% of the population, no related adverse effects have been detected in the many recipients of the HBsAg vaccine, which is manufactured in Baker's yeast (8). Use of yeast promoters with constitutive or high basal expression, like GPDp used for ApxIIA (37), or CUP1p used for ovalbumin (41), results in selection for reduced expression; consequently, transformants are unstable and store poorly (6). To avoid this, the GAL1p promoter, tightly suppressed in glucose media and highly induced by galactose if catabolite repression is first relieved by growth on a non-fermentable carbon source such as glycerol was used. The GFP bex1 ORF (2) was cloned by PCR into a series of GAL1p vectors (6, 13); expression in S. cerevisiae transformants was measured using a microtiter plate fluorimeter (ex 488 mM, em 520 nM).

The nucleotide and amino acid sequences of GFP bex1 OFR are as follows:

Nucleotide sequence:

(SEQ ID NO:10)

ATGAGTAAAGGAGAAGAACTTTTCACTGGAGTTGTCCCAATTCTTGTTGA
ATTAGATGGTGATGTTAATGGGCACAAATTTTCTGTCAGTGGAGAGGGTG
AAGGTGATGCAACATACGGAAAACTTACCCTTAAATTTATTTGCACTACT
GGAAAACTACCTGTTCCATGGCCAACACTTGTCACTACTTTCACTTATGG
TGTTCAATGCTTTTCAAGATACCCAGATCATATGAAACAGCATGACTTTT
TCAAGAGTGCCATGCCCGAAGGTTATGTACAGGAAAGAACTATATTTTTC
AAAGATGACGGGAACTACAAGACACGTGCTGAAGTCAAGTTTGAAGGTGA
TACCCTTGTTAATAGAATCGAGTTAAAAGGTATTGATTTTAAAGAAGATG
GAAACATTCTTGGACACAAATTGGAATACAACTATAACTCACACAATGTA
TACATCATGGCAGACAAACAAAAGAATGGAATCAAAGCTAACTTCAAAAT
TAGACACAACATTGAAGATGGAAGCGTTCAACTAGCAGACCATTATCAAC
AAAATACTCCAATTGGCGATGGCCCTGTCCTTTTACCAGACAACCATTAC
CTGTCCACACAATCTGCCCTTTCGAAAGATCCCAACGAAAAGAGAGACCA
CATGGTCCTTCTTGAGTTTGTAACAGCTGCTGGGATTACACATGGCATGG
ATGAACTATACAAATAA
protein sequence

(SEQ ID NO:11)

MSKGEELFTGVVPILVELDGDVNGHKFSVSGEGEGDATYGKLTLKFICTT
GKLPVPWPTLVTTFTYGVQCFSRYPDHMKQHDFFKSAMPEGYVQERTIFF
KDDGNYKTRAEVKFEGDTLVNRIELKGIDFKEDGNILGHKLEYNYNSHNV
YIMADKQKNGIKANFKIRHNIEDGSVQLADHYQQNTPIGDGPVLLPDNHY
LSTQSALSKDPNEKRDHMVLLEFVTAAGITHGMDELYK

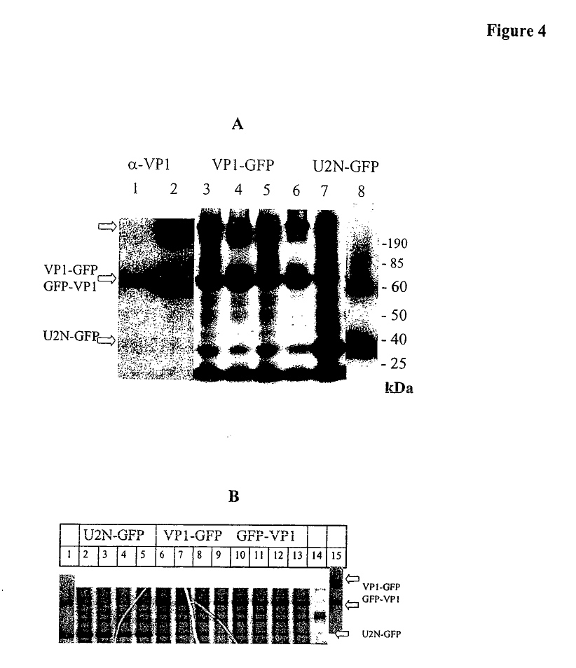

Highest GFP expression was obtained using the hybrid GAL1-CYC1p promoter in the YEp URA3 leu2d vector pPAP1466 in strain PAP1502 (31). Growth in the absence of uracil (Ura D/O medium) selects for a vector copy number of 15-20. However, in media lacking leucine (Leu D/O), this is increased at least 10 fold due to the very weak leu2d promoter. A proportional increase in GAL1p driven expression requires the high galactose-induced levels of the Gal4p activator provided by the integrated pPAP1488 plasmid in strain PAP1502 (31). Conditions were established giving reproducible GFP levels of 3-6% of total protein. Purified GST-GFP (FIG. 4B) served as a standard. Levels were increased to 6-12% by two vector modifications: insertion of the GAL1-GDH promoter (4), and insertion of two copies of the PGK terminator (6), producing vector pG2-GFP (FIG. 2A).

### Example 2

### YCP Preparation

Yeast cells at 2.10⁹/ml, at 25° C. in the absence of buffer, were equilibrated to 45° C. and then pH was adjusted to 11.8 (equivalent to 12.0 at 25° C.) with NaOH. The pH started to drop rapidly after 30-60 sec, and additional NaOH was added to keep the pH at 11.8 over the following 5-6 min, during which the cells became permeable to the alkali. Subsequent pH was stable and the extraction was terminated after 10-15 min by the addition of sufficient 1M Tris/HCl to bring the pH down to 6-8. The YCP's were then washed extensively with PBS and analysed for antigen retention and glucan exposure.

### Example 3

### Use of Concanavalin A-Alexafluor 594 (conA-594) to Measure Mannan Loss During YCP Extraction

To produce YCP's with exposed glucan, promoting M cell, DC and macrophage uptake, the overlying yeast cell wall manno-protein was extracted with alkali. Mannan extraction was monitored, by fluorimetric analysis, after staining YCP's with fluorescently labeled conA-594, or by FACS analysis after staining with the conA-647. Neither fluorescent lectin conjugate (Molecular Probes) has spectral overlap with GFP. ConA-488 whose fluorescence does overlap with GFP, was used for YCP's not expressing GFP. ConA is a lectin that binds selectively to mannan. Positive and negative controls were intact and mannan-stripped yeast cell walls produced by harsh alkaline extraction. Low background in the negative controls, which retain glucan, demonstrated selectivity of the conA conjugates for mannan. Duplicate analyses were reproducible +/−5%. Mild alkaline YCP extraction conditions were established (see Example 2) resulting in immediate killing of yeast cells and a 10 to 60% reduction in conA-594 binding, a range predicted to result in functional YCP vaccines.

### Example 4

### Use of Anti-β-Glucan Antibody to Measure Glucan Exposure During YCP Extraction and Uptake by 3T3-D1 Cells to Assess the Resultant Effect on Dectin1-Dependent Uptake

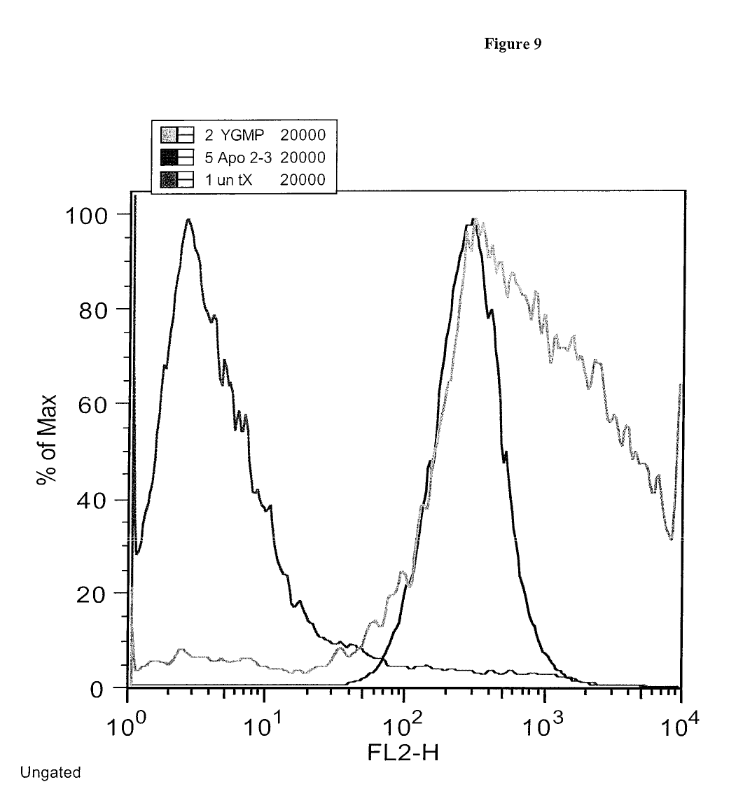

The extent of β-glucan exposure in YCP's determines the efficiency of translocation from the GI tract by M cells and will also determine the efficiency of subsequent uptake by APC's dependent on the dectin-1 and TLR2 β-glucan receptors, essentially controlling the adjuvant effect and immune response bias to a YCP vaccine. β-glucan exposure will also determine the efficiency of interactions with these same receptors in APCs after parenteral administration, and, therefore, of efficacy of vaccine delivery and glucan-dependent adjuvant activity. The most quantitative assay is provided by fluorescence-based FACS analysis of binding of an anti-β-glucan monoclonal antibody (FIG. 9). Binding is detected using a phyco-erythrin-labelled goat-anti mouse secondary antibody. As shown, untreated cells give a very small signal in the 2-8 range and YGMP particles, yeast cell walls almost entirely stripped of mannan (top left), give a broad peak in the 200-2000 range. Several different YCP preparations expressing U2N-Apo A1 gave peaks in the 100-500 range, a 20-100 fold increase in binding.

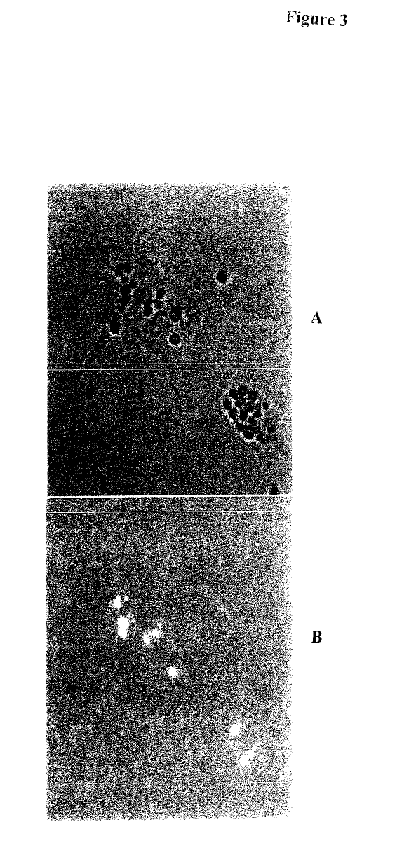

A direct measure of dectin-1-dependent uptake is provided by murine 3T3 cells expressing dectin-1 (47). Murine 3T3 cells do not normally phagocytose yeast. Expression of the dectin-1 receptor protein is sufficient to allow 3T3-D1 phagocytosis of yeast cells with exposed β-glucan. The background level of about 20% seen in unprocessed fresh cells probably reflects the small amounts of glucan exposed at bud scars. Processing of cells to produce YCP's increases uptake up to the 90% level seen for yeast cell walls almost entirely stripped of mannan. Uptake of YCPs by J774 macrophages (47) was also tested to assess the efficacy of phagocytosis in the presence of the full array of APC receptors. (FIG. 3).

### Example 5

### Testing Polymeric Fusion Partners (Scaffolds) for Selective Antigen Retention During YCP Extraction

Even under these mild YCP extraction conditions (see Example 2), 95% of the GFP, a compact soluble protein, was released from PAP1502 cells expressing pG2-GFP (Table 2). Naturally oligomeric or aggregated forms of protein antigens are superior immunogens, e.g., (45), and should also resist extraction.

The HB core antigen forms very stable VLP's, and GFP, flanked by flexible linkers (GGGGSGGGGT (SEQ ID NO: 12)) and fused between residues 78 and 80, at an external loop of a C-terminally truncated HBV core protein gene (codons 1-149)(FIG. 2B) retains fluorescence, indicative of normal folding (44). To test the potential of VLPs as antigen scaffolds, the gene encoding this fusion was cloned by PCR and inserted into pG2. The nucleotide and amino acid sequences of this fusion protein are as follows.

Nucleotide sequence:

(SEQ ID NO:13)

ATGGATATCGATCCTTATAAAGAATTCGGAGCTACTGTGGAGTTACTCTC
GTTTCTCCCGAGTGACTTCTTTCCTTCAGTACGAGACCTTCTGGATACCG
CCAGCGCGCTGTATCGGGAAGCCTTGGAGTCTCCTGAGCACTGCAGCCCT
CACCATACTGCCCTCAGGCAAGCAATTCTTTGCTGGGGGGAGCTCATGAC
TCTGGCCACGTGGGTGGGTGTTAACCTCGAGGATGGTGGAGGTGGCTCCG
GAGGGGGTGGTACCATGAGCAAGGGCGAGGAACTGTTCACTGGCGTGGTC
CCAATTCTCGTGGAACTGGATGGCGATGTGAATGGGCACAAATTTTCTGT
CAGCGGAGAGGGTGAAGGTGATGCCACATACGGAAAGCTCACCCTGAAAT
TCATCTGCACCACTGGAAAGCTCCCTGTGCCATGGCCAACACTGGTCACT
ACCCTCACCTATGGCGTGCAGTGCTTTTCCAGATACCCAGACCATATGAA
GCAGCATGACTTTTTCAAGAGCGCCATGCCCGAGGGCTATGTGCAGGAGA
GAACCATCTTTTTCAAAGATGACGGGAACTACAAGACCCGCGCTGAAGTC
AAGTTCGAAGGTGACACCCTGGTGAATAGAATCGAGCTGAAGGGCATTGA
CTTTAAGGAGGATGGAAACATTCTCGGCCACAAGCTGGAATACAACTATA
ACTCCCACAATGTGTACATCATGGCCGACAAGCAAAAGAATGGCATCAAG
GTCAACTTCAAGATCAGACACAACATTGAGGATGGATCCGTGCAGCTGGC
CGACCATTATCAACAGAACACTCCAATCGGCGACGGCCCTGTGCTCCTCC
CAGACAACCATTACCTGTCCACCCAGTCTGCCCTGTCTAAAGATCCCAAC
GAAAAGAGAGACCACATGGTCCTGCTGGAGTTTGTGACCGCTGCTGGGAT
CACACATGGCATGGACGAGCTGTACAAGGGTGGAGGTGGCTCCGGAGGGG
GTGGATCTAGAGACCTGGTAGTCAGTTATGTCAACACTAATATGGGTTTA
AAGTTCAGGCAACTCTTGTGGTTTCACATTAGCTGCCTCACTTTCGGCCG
AGAAACAGTTATAGAATATTTGGTGTCTTTCGGAGTGTGGATCAGAACTC
CTCCAGCTTATAGGCCTCCGAATGCCCCTATCCTGTCGACACTCCCGGAG
ACTACGGTAGTA
Protein sequence:

(SEQ ID NO:14)

MDIDPYKEFGATVELLSFLPSDFFPSVRDLLDTASALYREALESPEHCSP
HHTALRQAILCWGELMTLATWVGVNLEDGGGGSGGGGTMSKGEELFTGVV
PILVELDGDVNGHKFSVSGEGEGDATYGKLTLKFICTTGKLPVPWPTLVT
TLTYGVQCFSRYPDHMKQHDFFKSAMPEGYVQERTIFFKDDGNYKTRAEV
KFEGDTLVNRIELKGIDFKEDGNILGHKLEYNYNSHNVYIMADKQKNGIK
VNFKIRHNIEDGSVQLADHYQQNTPIGDGPVLLPDNHYLSTQSALSKDPN
EKRDHMVLLEFVTAAGITHGMDELYKGGGGSGGGGSRDLVVSYVNTNMGL
KFRQLLWFHISCLTFGRETVIEYLVSFGVWIRTPPAYRPPNAPILSTLPE
TTVV

Maximal levels of fluorescence were only 7% of those shown by pG2-GFP. However, 80% of this fluorescence was retained in YCP's after extraction that removed 30% of the mannan, demonstrating the anticipated selective retention of a VLP antigen fusion.

TABLE 2
Retention of GFP fusion fluorescence in YCP's with decreasing
residual mannan content. These are the averages of data from
several YCP extractions.

| % initial mannan content | | | | |
|---|---|---|---|---|
| (ConA 594 fluorescence) | GFP | VPI-GFP | GFP-VP1 | U2N-GFP |
| 80 | 5 | 85 | 80 | 70 |
| 70 | <1 | 75 | 70 | 55 |
| 60 | | 70 | | 40 |
| 40 | | 55 | 45 | 25 |

### Example 6

### The Hepatitis B (HBsAg) VLP Scaffold

Since direct C- or N-terminal antigen-capsid fusions would be more generally useful than the internal HBc fusion, N- or C-terminal fusions of GFP to two different viral capsids were generated and tested. First, because an N-terminal GFP-HBsAg fusion can be incorporated into functional HB virions, with some of the GFP exposed on the surface (20), and N-terminal fusions to HBsAg have previously been used for antigen presentation (48), the HBsAg reading frame used in Engerix B was cloned into pG2-GFP to produce GFP-HBsAg (FIG. 2C). The nucleotide and amino acid sequences of the HBsAg (GenBank Accession No. HBAJ3116) used are as follows:

Nucleotide sequence:

(SEQ ID NO:17)

ATGGAGAACATCACATCAGGATTCCTAGGACCCCTGCTCGTGTTACAGGC
GGGGTTTTTCTTGTTGACAAGAATCCTCACAATACCGCAGAGTCTAGACT
CGTGGTGGACTTCTCTCAATTTTCTATGGGGATCTCCCGTGTGTCTTGGC
CAAAATTCGCAGTCCCCAACCTCCAATCACTCACCAACCTCCTGTCCTCC
AATTTGTCCTGGTTATCGCTGGATGTGTCTGCGGCGTTTTATCATATTCC
TCTTCATCCTGCTGCTATGCCTCATCTTCTTATTGGTTCTTCTGGATTAT
CAAGGTATGTTGCCCGTTTGTCCTCTAATTCCAGGATCAACAACAACCAG
TACGGGACCATGCAAAACCTGCACGACTCCTGCTCAAGGCAACTCTATGT
TTCCCTCATGTTGCTGTACAAAACCTACGGATGGAAATTGCACCTGTATT
CCCATCCCATCGTCCTGGGCTTTCGCAAAATACCTATGGGAGTGGGCCTC
AGTCCGTTTCTCTTGGCTCAGTTTACTAGTGCCATTTGTTCAGTGGTTCG
TAGGGCTTTCCCCCACTGTTTGGCTTTCAGCTATATGGATGATGTGGTAT
TGGGGGCCAAGTCTGTACAGCATCGTGAGTCCCTTTATACCGCTGTTACC
AATTTTCTTTTGTCTTTGGGTATACATT
Protein sequence:

(SEQ ID NO:18)

MENITSGFLGPLLVLQAGFFLLTRILTIPQSLDSWWTSLNFLWGSPVCLG
QNSQSPTSNHSPTSCPPICPGYRWMCLRRFIIFLFILLLCLIFLLVLLDY
QGMLPVCPLIPGSTTTSTGPCKTCTTPAQGNSMFPSCCCTKPTDGNCTCI
PIPSSWAFAKYLWEWASVRFSWLSLLVPFVQWFVGLSPTVWLSAIWMMWY
WGPSLYSIVSPFIPLLPIFFCLWVYI

This direct fusion failed to express GFP fluorescence, so a flexible peptide linker (GGTSGGSTGLSSG (SEQ ID NO: 6)) was inserted between GFP and HBsAg, restoring fluorescence. Maximal fluorescence of the HBsAg fusion in strain PAP1502 was only 10% of G2-GFP (FIG. 2). The HBsAg fusion has not been studied further, since expression of the VP1 fusions was much stronger.

### Example 7

### The Polyoma VP1 VLP Scaffold

VLP's of the polyoma virus VP1 capsid assemble stably in the yeast cytoplasm (35) and VP1 fusions have been used for antigen presentation (43). The VP1 gene of mouse polyoma virus strain A2 (42, 46), was cloned by PCR directly from an A2 viral isolate. The nucleotide and amino acid sequences of this VP1 protein are as follows (open reading frame starts at base 10 after the Xho 1 site):

Nucleotide sequence:

(SEQ ID NO:19)

CTC GAG AAG ATG GCC CCC AAA AGA AAA AGC GGC GTC
TCT AAA TGC GAG ACA AAA TGT ACA AAG GCC TGT CCA
AGA CCC GCA CCC GTT CCC AAA CTG CTT ATT AAA GGG
GGT ATG GAG GTG CTG GAC CTT GTG ACA GGG CCA GAC
AGT GTGACAGAAATAGAAGCTTTTCTGAACCCCAGAATGGGGCAGCCAC
CCACCCCTGAAAGCCTAACAGAGGGAGGGCAATACTATGGTTGGAGCAGA
GGGATTAATTTGGCTACATCAGATACAGAGGATTCCCCAGAAAATAATAC
ACTTCCCACATGGAGTATGGCAAAGCTCCAGCTTCCCATGCTCAATGAGG
ACCTCACCTGTGACACCCTACAAATGTGGGAGGCAGTCTCAGTGAAAACC
GAGGTGGTGGGCTCTGGCTCACTGTTAGATGTGCATGGGTTCAACAAACC
CACAGATACAGTAAACACAAAAGGAATTTCCACTCCAGTGGAAGGCAGCC
AATATCATGTGTTTGCTGTGGGCGGGGAACCGCTTGACCTCCAGGGACTT
GTGACAGATGCCAGAACAAAATACAAGGAAGAAGGGGTAGTAACAATCAA
AACAATCACAAAGAAGGACATGGTCAACAAAGACCAAGTCCTGAATCCAA
TTAGCAAGGCCAAGCTGGATAAGGACGGAATGTATCCAGTTGAAATCTGG
CATCCAGATCCAGCAAAAAATGAGAACACAAGGTACTTTGGCAATTACAC
TGGAGGCACAACAACTCCACCCGTCCTGCAGTTCACAAACACCCTGACAA
CTGTGCTCCTAGATGAAAATGGAGTTGGGCCCCTCTGTAAAGGAGAGGGC
CTATACCTCTCCTGTGTAGATATAATGGGCTGGAGAGTTACAAGAAACTA
TGATGTCCATCACTGGAGAGGGCTTCCCAGATATTTCAAAATCACCCTGA
GAAAAAGATGGGTCAAAAATCCCTATCCCATGGCCTCCCTCATAAGTTCC
CTTTTCAACAACATGCTCCCCCAAGTGCAGGGCCAACCCATGGAAGGGGA
GAACACCCAGGTAGAGGAGGTTAGAGTGTATGATGGGACTGAACCTGTAC
CGGGGGACCCTGATATGACGCGCTATGTTGACCGCTTTGGAAAAACAAAG
ACTGTATTTCCTGGAAATTAAGATCTGCC
Amino acid sequence:

(SEQ ID NO:20)

MAPKRKSGVSKCETKCTKACPRPAPVPKLLIKGGMEVLDLVTGPDSVTEI
EAFLNPRMGQPPTPESLTEGGQYYGWSRGINLATSDTEDSPENNTLPTWS
MAKLQLPMLNEDLTCDTLQMWEAVSVKTEVVGSGSLLDVHGFNKPTDTVN
TKGISTPVEGSQYHVFAVGGEPLDLQGLVTDARTKYKEEGVVTIKTITKK
DMVNKDQVLNPISKAKLDKDGMYPVEIWHPDPAKNENTRYFGNYTGGTTT
PPVLQFTNTLTTVLLDENGVGPLCKGEGLYLSCVDIMGWRVTRNYDVHHW
RGLPRYFKITLRKRWVKNPYPMASLISSLFNNMLPQVQGQPMEGENTQVE
EVRVYDGTEPVPGDPDMTRYVDRFGKTKTVFPGN

Insertion into pG2-GFP produced both N- and C-terminal GFP fusions (FIG. 2D, E). In both cases, while the direct GFP fusion failed to express fluorescence, insertion of a flexible peptide linker allowed normal fluorescence development. Maximal fluorescence of the two VP1 fusions in strain PAP1502 was then 35 and 40% of G2-GFP, respectively (FIG. 2). Western blots confirmed the presence of GFP-VP1 and VP1-GFP fusions of the expected 70 kDa size (FIG. 4A, lanes 1 and 2, respectively), together with a high molecular weight band (upper arrow) found only in VP1-GFP cells that probably corresponds to VP1-GFP pentamers (35). Stained gels (FIG. 4A, lanes 3-6) showed these same species at levels consistent with fluorescence data, indicating that GFP constituted about 4% of total protein.

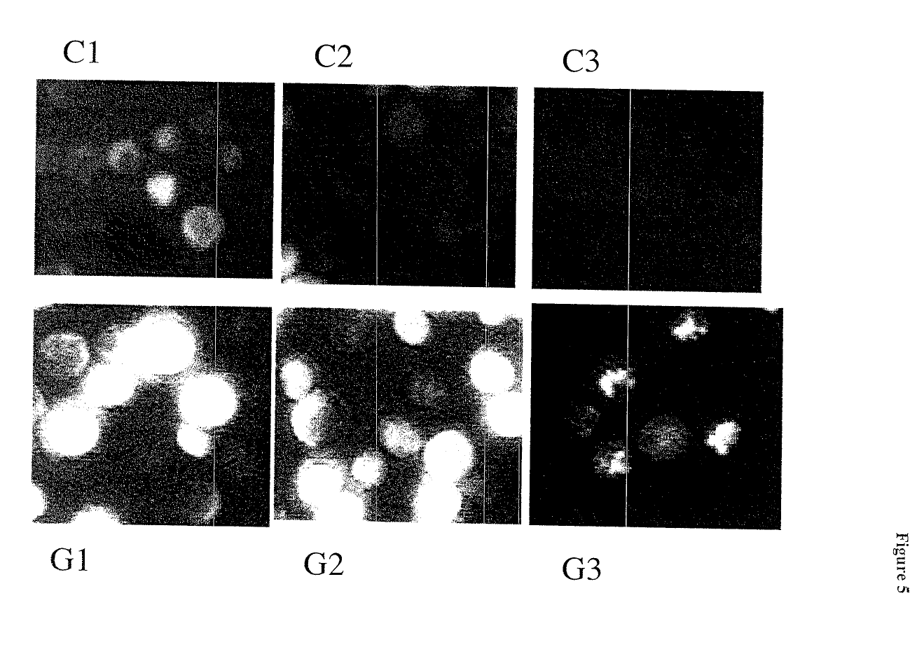

The VP1-GFP fusion was chosen for further study because of high expression and the prediction from VP1 structure (27) that C-terminal fusions should decorate the VLP surface (at pentamer intersections), so that larger antigens should also be acceptable at this fusion site. Microscopy of cells containing VP1-GFP showed the presence of large intracellular fluorescent aggregates (FIG. 5). In broken cells, 97% of the VP1 fusions were found in a 100 kg pellet, consistent with presence in VLP's. Retention of these VP1 fusions in YCP's with 20 to 60% mannan loss was 85 to 55% (Table 2), demonstrating selective retention of the VP1-GFP antigen during YCP vaccine production. The GFP-VP1 fusion had only slightly reduced stability (Table 2). Retention of both VP1-GFP and GFP-VP1 during YCP extraction is evident from stained gels (FIG. 4B, lanes 6-13).

ConA-594 fluorescent staining of cells expressing VP1-GFP (FIG. 5 C1-3) visually confirmed the quantitative fluorescence data, showing a corresponding decrease in stain intensity with time of extraction. The GFP signal was too strong to reveal detail except in cells retaining only about 20% of both mannan and GFP. The VP1-GFP was then seen as several discrete punctate aggregates (FIG. 5 G3).

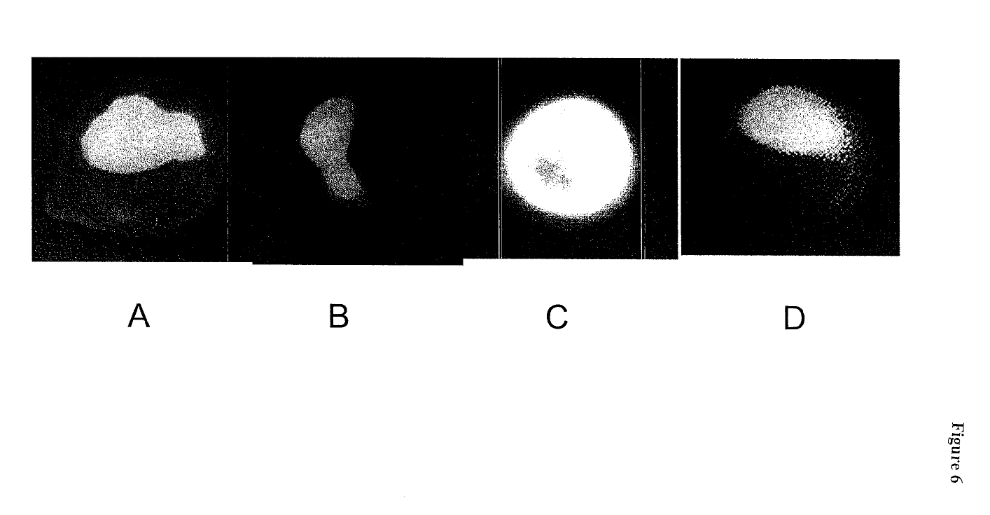

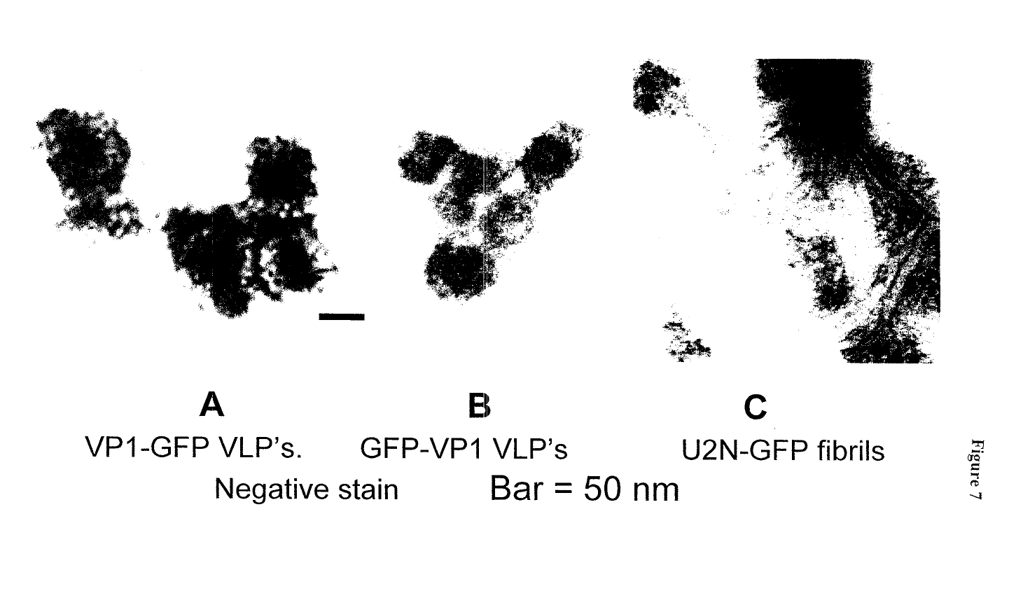

Digitally sectioned micrographs of single ConA-594-stained cells expressing VP1-GFP showed large aggregates clearly contained within the confines of the cell wall (FIG. 6A). In YCP's with 60% residual mannan, a decrease in both signals is seen (FIG. 6B). The high speed pellet fraction of these cells broken in the presence of 40 mM octyl glucoside was highly enriched for approximately 60 nM VLP's, as shown by negatively stained electron micrographs (FIG. 7A). A similar fraction from cells expressing GFP-VP1 Showed VLP's of similar size but with less distinct outlines (FIG. 7B).

### Example 8

### The U2N Scaffold

U2N, a 65-residue N-terminal peptide from the yeast enzyme Ure2p, is the smallest and best characterized asparagine-glutamine (NQ)-rich, self-aggregating yeast peptide. It spontaneously aggregates into stacked cross-β sheet fibrils when over-expressed from the fully induced GAL1p promoter (10, 32). GFP and other proteins C-terminally fused to U2N decorate the surface of the fibril in normally folded and functional forms (3). U2N was cloned by PCR from pH324 (10) and inserted into pG2-GFP, producing U2N-GFP. The nucleotide and amino acid sequences of U2N codons 1-76 and a flexible linker, all optimized for yeast expression, are as follows:

Nucleotide sequence:

(SEQ ID NO:21)

ATGATGAATAACAACGGCAACCAAGTGTCGAATCTCTCCAATGCGCTCCG
TCAAGTAAACATAGGAAACAGGAACAGTAATACAACCACCGATCAAAGTA
ATATAAATTTTGAATTTTCAACAGGTGTAAATAATAATAATAATAACAAT
AGCAGTAGTAATAACAATAATGTTCAAAACAATAACAGCGGCCGCTCGAG
C
Amino acid sequence:

(SEQ ID NO:22)

MMNNNGNQVSNLSNALRQVNIGNRNSNTTTDQSNINFEFSTGVNNNNNNN
SSSNNNNVQNNNSGRSS

Fluorescence in induced PAP1502 cells was reduced by 40% relative to pG2-GFP (FIG. 2), possibly because of the high proportion of rare codons in U2N. Western blots using anti-GFP showed a band of the predicted 34 kDa size (FIG. 4A, lane 8), also readily visible as a prominent band (4-5% of total protein) following coomassie staining (FIG. 4A, lanes 7). In broken cells, 98% of the U2N-GFP was found in a 14,000 g pellet, indicating presence in very large aggregates. In micrographs, these were seen proximal to the plasma membrane in cells expressing U2N-GFP (FIG. 6C). In YCP's with 60% residual mannan, a decrease in both signals is seen (FIG. 6D). U2N-GFP aggregates in YCP's were not affected by extraction with 1% triton X100; this could, therefore, be used to remove additional YCP non-antigen proteins. A 5 to 14,000 g pellet from these YCP's was enriched for fibrillar structures, as shown in FIG. 7C. Retention of fluorescence of the U2N-GFP fusion in YCP's with 20-60% mannan loss was 70-25% (Table 2), demonstrating selective retention, though not to the extent shown by VP1-GFP. Essentially complete retention of the U2N-GFP fusion protein is evident from stained gels (FIG. 4B, lanes 2-5).

### Example 9

### Stability of Transformants and Proteins

pG2-GFP-VP1, -VP1-GFP and -U2N-GFP transformants of strain PAP1502 were stable indefinitely after growth in glucose media, as shown by induced GFP expression levels. The high levels of GFP fusions in induced cells, measured by fluorescence and by band intensity of total proteins on stained SDS gels (FIG. 4), were stable for several days at 4° C. and indefinitely when frozen at −75° C. in 15% glycerol. Fusion proteins in YCP vaccines were stable indefinitely when lyophilized or when frozen at −20° C. in PBS.

### Example 10

### YCP Uptake in Tissue Culture

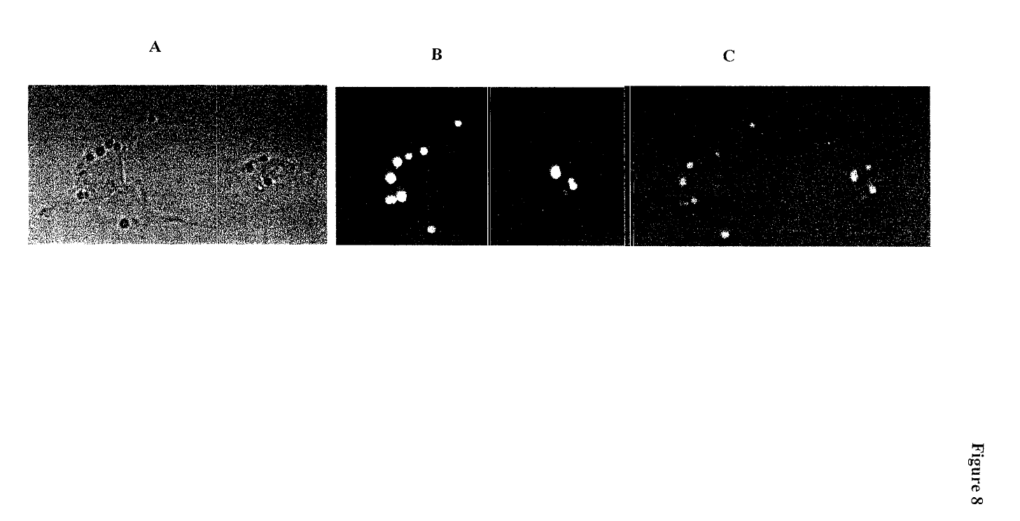

YCP's expressing VP1-GFP and retaining 80% of their initial mannan were avidly phagocytosed by J774 macrophages and also by 3T3-D1 fibroblasts (47), which largely ignored intact yeast cells, demonstrating the effect of glucan exposure on YCP uptake. Internalized YCP's were clearly visible by GFP fluorescence or by staining of cell wall glucan with congo red (18) (FIG. 8). An identical sample of YCP's containing VP1-GFP but unstained was invisible with the filter used for congo red, so GFP did not contribute to the signal. While the GFP signal decreased in J774 cells by more than 80% by 24 hours following phagocytosis, presumably due to proteolysis of the scaffolded antigen, the congo-red stained cell walls were stable for much longer.

### Example 11

### YCP VP1-GFP Vaccine Function

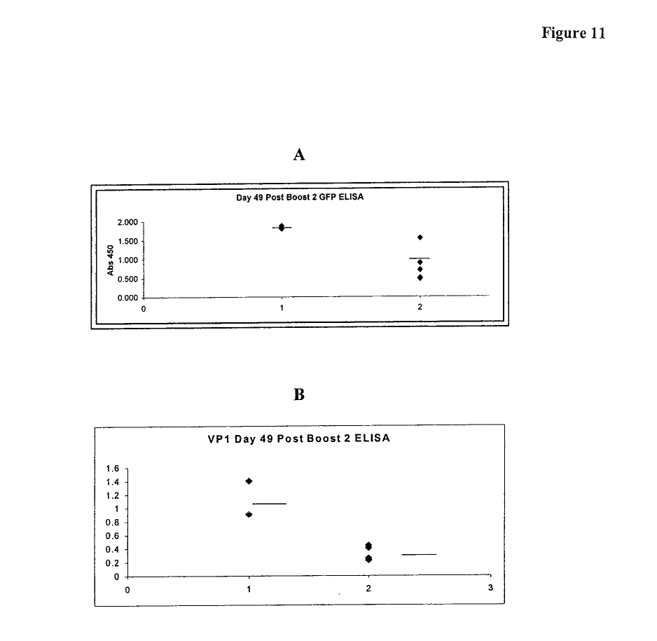

Groups of 5 mice were vaccinated with 4.10⁸ YCP's of the VP1-GFP formulation with 80% residual mannan, estimated to contain about 15 μg of VP1-GFP. Pre-bleeds provided negative controls. All positive control mice receiving this YCP dose ip produced strong serum IgG responses to both VP1 and GFP one week after a single boost. Four of the 5 mice receiving vaccine by oral gavage produced a significant serum IgG response to VP1 after a single boost, and all five demonstrated a strong response to GFP and a weaker but clearly significant response to VP1 10 days after a second boost (FIG. 11).

### Example 12

### YCP U2N-GFP Vaccine Function

In a separate experiment, groups of 5 C57/B6 mice were vaccinated, either orally or ip, with 4.108 YCP's of a U2N-GFP vaccine formulation, also retaining about 80% of the original cell wall mannan, and containing 10-15 μg of U2N-GFP per dose. As for the VPI-GFP vaccine, mice were bled 10 days after the second boost. All mice vaccinated ip produced IgG responses to GFP greater than 1/1600 while the 5 orally vaccinated mice produced IgG responses to GFP in the range from 1/100 to 1/800, demonstrating an average response at least a strong as to the VP1-GFP vaccine.

These oral vaccines were not protected from stomach digestion, so that responses to oral and ip doses cannot be directly compared. In addition, response to oral vaccines is usually reported to require multiple boosts (11, 34, 37).

These results suggest that YCP dosage and adjuvant activity is sufficient to circumvent oral tolerance (23, 25, 26) and establish both oral and parenteral efficacy of prototype YCP vaccines using either VP1 VLP's or U2N as scaffold for the GFP model antigen.

9 months after the final doses of YCP VPI-GFP vaccine, orally vaccinated mice showed no residual serum IgG while IP-vaccinated mice still showed titers of 1/200 to > 1/800. These mice were tested for protection against infection by polyoma virus expressing the same capsid by RT-PCR analysis of viral DNA loads in multiple tissues five days after infection, when titers are normally maximal. While orally vaccinated mice showed no evidence of protection, all ip vaccinated mice tested were highly protected, such that viral DNA titers in all tissues were reduced at least 1000 fold relative to non-vaccinated controls.

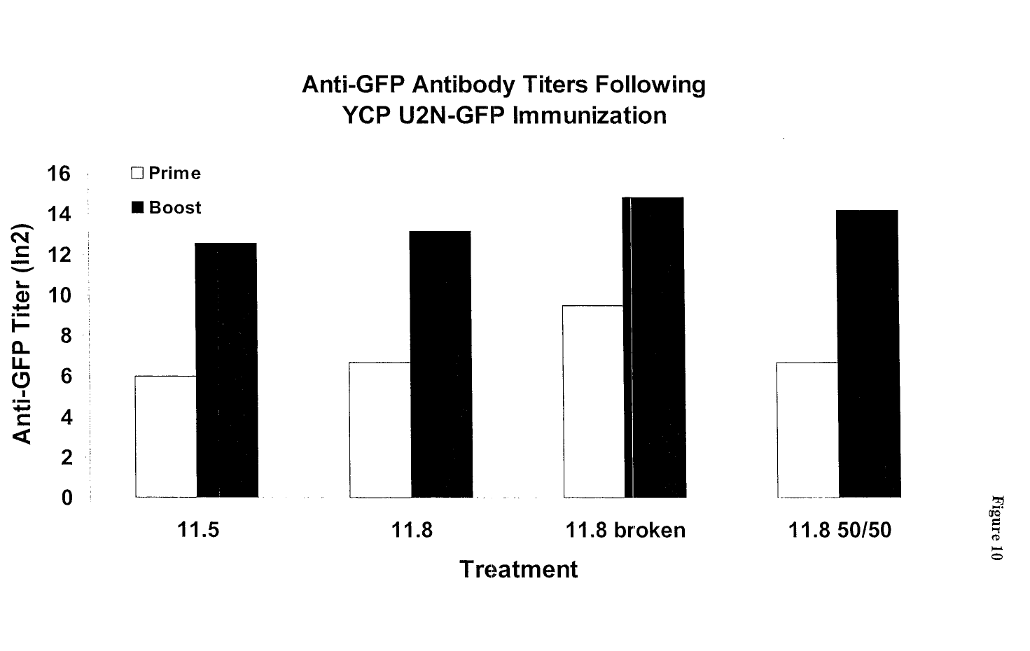

In a separate experiment, YCP U2N-GFP vaccines were administered at 2 week intervals to C57/B6 mice by subcutaneous dosage using 25% of the number of particles used orally. The YCPs were administered either intact or after bead-breakage. The results of serum IgG assays by ELISA showed that the broken YCPs were a superior immunogen by this route. Titers after a single dose were 100-12,000, reached 12,000-200,000 after a single boost and reached a maximum of 500,000 to 2,000,000 after 2-3 boosts (FIG. 10).

### Example 13

### Primers Used in the Preparation of the Constructs Described Above

A. Murine Polyoma Virus Strain A2 VP1 Using DNA from Infected Mouse Sera as Template

The purpose of ODT522A-3A is to clone the polyoma strain A2 VP1 antigen as an 1160 bp Xba1/Xho1-Bgl2 PCR fragment for insertion into vectors such as pG2, making pG2-VP1, providing Gal-inducible expression of VP1 VLP's. Homology=TM of 60-65 C

ODT 522A Xba1 Xho1 Polyoma VP1 ORF→
5′ CGGC TCT AGA CTC GAG GAAG ATG GCC CCC AAA AG
ODT 523A → Polyoma VP1 ORF Bgl2
5′ CA AAG ACT GTA TTT CCT GGA AAT TAA TGA GAT
CTGCC
3′ GT TTC TGA CAT AAA GGA CCT TTA ATT ACT CTA
GACGG
Reverse and complement
5′ GGC AGA TCT CAT TAA TTT CCA GGA AAT ACA GTC
TTTG

B. GFP Bex from the Gene Cloned in pDJ388
ODT558. GFP (bex) Sense 48mer. Use with 559 to clone GFP, using pDJ388 as template, for insertion downstream of Ure2N in yeDP60 cut Not 1+R1.

Also can use to clone in Ure2N-VP1-pB4 as Xho-Bgl2, replacing VP1

```
                      Not1 Xho1 GFP-bex→
GC TGC TGC GGC CGC TCG AGC ATG AGT AAA GGA GAA GAA CTT TTC ACT G
              Ser Gly Arg Ser Ser Met Ser Lys Gly Glu Glu Leu Phe Thr
```

ODT559. GFPbex anti-sense, use with 558 to clone GFP from pDJ388

GFP C-term → Bgl2 EcoR1
5′ CAT GGC ATG GAT GAA CTA TAC AAA TAA GAT CTG AAT TCA GCA GC
3′ GTA CCG TAC CTA CTT GAT ATG TTT ATT CTA GAC TTA AGT CGT CG
Reverse-complement EcoR1 Bgl2
559 5′ GCT GCT GAA TTC AGA TCT TAT TTG TAT AGT TCA TCC AGT CCA TG

C. Sub-Cloning Strain A2 VP1 from pG2-VP1 into pG2-GFP, Producing a C-Terminal VP1-GFP Fusion with a Flexible Linker

ODT579A, Primes from the BamH1 site at the fusion of GAL and GDH promoter fragments in pG2. Use with 578 to clone GDH-VP1.

5′ GA TCG TCG ACG GAT CCC CAG

ODT585 VP1 C-term anti-sense primer (Xba1-Sal1). Use with 579 to clone GDH-VP1 as a Bam to Sal1 fragment for insertion into pG2-GFP (cut Bam-Xho). The Xho and Xba (or Spe1) sites flanking the VP1 sequence could then be used to insert other antigens.

```
VP1 → Xba1 Spe1
CA AAG ACT GTA TTT CCT GGA AAT CTA GAC GGA ACT AGT
GT TTC TGA CAT AAA GGA CCT TTA GAT CTG CCT TGA TCA
        K T V F P G N L D G T S
                                                            Linker →
                                  Sal1
GGT GGA AGT GGG TCG ACG GTC CTG G
CCA CCT TCA CCC AGC TGC CAG GAC C
  G G S G S T
Reverse and complement = 585 63 mer,
5′ CCA GGA CCG TCG ACC CAC TTC CAC CAC TAG TTC CGT
CTA GAT TTC CAG GAA ATA CAG TCT TTG
```

D. ODT586. Anti-sense primer for use with ODT579A (above) for cloning GFP, preceded by the GDH promoter fragment from pG2-GFP, for N-terminal fusion to VP1 or HbsAg with a flexible linker.

```
GFP → Spe1
GGC ATG GAT GAA CTA TAC AAA GGA GGT ACT AGT GGT
CCG TAC CTA CTT GAT ATG TTA CCT CCA TGA TCA CCA
  G M D E L Y K G G T S G
                                  Xba1 Sal1 Linker →
GGA AGT ACT GGT CTA GAG TCG ACG GTC CTG G
CCT TCA AGC CCA GAT CTC AGC TGC CAG GAC C
  G S T G L E S T
Reverse and complement = 585 67 mer,
5′ CCA GGA CCG TCG ACT CTA GAC CAG TAC TTC CAC CAC
TAG TAC CTC CTT TGT ATA GTT CAT CCA TGC C
```

E. Primers to Clone Ure2N from the URE2 Gene Cloned in pH324.
Purpose of ODT534-535 is to clone Ure2p codons 1-65 as a 215 bp Sal1/Nhe1 to Xho1 fragment for insertion into YEp-Gal-VP1 cut Xho1/CIP upstream of VP1.

The Ure2-fragment will be in-frame with the VP10RF:

GGC CGC TCG AGA ATG
Gly Arg Ser Ser Met (VP1)

Any other antigen can then be cloned in frame with Ure2N as an Xho1-Bgl2 fragment, replacing VP1, using the same frame: GGC TCG AGA-(ATG etc).

```
534 Ure2-N sense 42 mer
                          Sal1 Nhe1
5′ GGA TGC GTC GAC GCT AGC AGA ATG ATG AAT AAC AAC
GGC AAC
557 Ure2-N anti-sense Not1 Xho1 EcoR1
5′ CAA AAC AAT AAC AGC GGC CGC TCG AGA ATT CAG
CAGC
3′ GTT TTG TTA TTG TCG CCG GCG AGC TCT TAA GTC
      Gln Asn Asn Asn Ser Gly Arg Ser (Arg)
GTCG
Reverse and complement, 31 mer
557 = 5′ GCT GCT GAA TTC TCG AGC GGC CGC TGT TAT TGT TTTG
```

F, Primers for cloning HbsAg from vector GA5 (pGAL-Hbs-tADH1) isolated from total DNA of ATCC strain 20705 as an Xho1 to Bgl2 fragment for insertion into pGAL vectors.

ODT520 Hbs-Sense 30mer
5′ GGG CTC GAG AAT GGA GAA CAT CRC ATC AGG
ODT521 Hbs Anti-sense, C-terminus 33mer
5′ C TTT TGT CTT TGG GTA TAC ATT TAAGATCTCCC
Rev + comp
5′ GGG AGA TCT TAA ATG TAT ACC CAA AGA CAA AAG

G. Primers for Cloning eGFP-HBc from pET28a2-c149eGFP into pG2.

ODT587 sense. Use with 591 to clone eGFP-HbcAg as a Spe1 to Bgl2 fragment for cloning into pG2-GFP after converting its Xho1 site to Spe1 using ODT589.

```
                          Spe1 HBc →
5′ GGG AGC ACT AGT AAG ATG GAT ATC GAT CCT TAT AAA
                                              M D I D P Y K
G
ODT589 Xho1 to Spe1
    (Xho1) Spe1
5′ TCGACGACTAGTCG
              GCTCATGAGCAGCT
ODT591 eGFP-HBc anti-sense C-terminal primer
        L S T L P E T T V V Stop Bgl2
5′ CTG TCG ACA CTC CCG GAG ACT ACG GTA GTA TAA GAT
CTC GCG CC
      GAC AGC TGT GAG GGC CTC TGA TGC CAT CAT ATT CTA
GAG CGC GG
Reverse and complement = 591 44mer
5′ GGC GCG AGA TCT TAT ACT ACC GTA GTC TCC GGG AGT
GTC GAC AG
```

Other Sequences:
1 The plasmid PAP 1488 is described in
Pedersen, P. A., J. H. Rasmussen, and P. L. Joorgensen. 1996. Expression in high yield of pig alpha 1 beta 1 Na,K-ATPase and inactive mutants D369N and D807N in Saccharomyces cerevisiae. J Biol Chem 271:2514-22.

2. The Y. pestis LcrV gene was cloned from plasmid pCD1, GenBank sequence YPCD1 using ODT603 and 604 as a 750 bp fragment, cut with Sal1 and Bgl2 for insertion into pG2-UNL-GFP cut Xho1+Bgl2 to produce pG2-UNL-LcrV

ODT603. LcrV Fwd 60mer Spe1 Sal1 LcrV →
5′ CGG CGA GCC ACT AGT GGT GGA AGT GGT TCG TCG ACC ATG ATT AGA
THR SER GLY GLY SER GLY SER SER Thr MET ILE ARG
GCC TAC GAA CAA AAC
ALA TYR GLU GLN ASN
ODT604. LcrV RevA LcrV → Bgl2
5′ G CTA GAT GAC ACG TCT GGT AAA TAA GAT CTC GCG CC
3′ C GAT CTA CTG TGC AGA CCA TTT ATT CTA GAG CGC GG
Reverse and complement = 604 36mer,
5′ GGC GCG AGA TCT TAT TTA CCA GAC GTG TCA TCT AGC

### REFERENCES

1. Allen, S. C., A. Byron, J. M. Lord, J. Davey, L. M. Roberts, and G. Ladds. 2005. Utilisation of the budding yeast Saccharomyces cerevisiae for the generation and isolation of non-lethal ricin A chain variants. Yeast 22: 1287-97.
2. Anderson, M. T., I. M. Tjioe, M. C. Lorincz, D. R. Parks, L. A. Herzenberg, G. P. Nolan, and L. A. Herzenberg. 1996. Simultaneous fluorescence-activated cell sorter analysis of two distinct transcriptional elements within a single cell using engineered green fluorescent proteins. Proc Natl Acad Sci USA 93:8508-11.
3. Baxa, U., V. Speransky, A. C. Steven, and R. B. Wickner. 2002. Mechanism of inactivation on prion conversion of the Saccharomyces cerevisiae Ure2 protein. Proc Natl Acad Sci USA 99:5253-60.
4. Bitter, G. A., and K. M. Egan. 1988. Expression of interferon-gamma from hybrid yeast GPD promoters containing upstream regulatory sequences from the GAL1-GAL10 intergenic region. Gene 69:193-207. Blander, J. M. and Medzhitov, R. 2006. TOll-dependent selection of microbial antigens for presentation by dendritic cells. Nature 440 808-812.
5. Brown, G. D., and S. Gordon. 2003. Fungal beta-glucans and mammalian immunity. Immunity 19:311-5.
6. Cartwright, C. P., Y. Li, Y. S. Zhu, Y. S. Kang, and D. J. Tipper. 1994. Use of beta-lactamase as a secreted reporter of promoter function in yeast. Yeast 10:497-508.
7. de la Rosa, G., M. Yanez-Mo, R. Samaneigo, D. Serrano-Gomez, L. Martinez-Munoz, E. Fernandez-Ruiz, N. Longo, F. Sanchez-Madrid, A. L. Corbi, and P. Sanchez-Mateos. 2005. Regulated recruitment of DC-SIGN to cell-cell contact regions during zymosan-induced human dendritic cell aggregation. J Leukoc Biol 77:699-709.
8. Dimiceli, L., V. Pool, J. M. Kelso, S. V. Shadomy, J. Iskander, and V. A. Team. 2005. Vaccination of yeast sensitive individuals: review of safety data in the US vaccine adverse event reporting system (VAERS). Vaccine. In press.
9. Dux, M. P., R. Barent, J. Sinha, M. Gouthro, T. Swanson, A. Barthuli, M. Inan, J. T. Ross, L. A. Smith, T. J. Smith, R. Webb, B. Loveless, I. Henderson, and M. M. Meagher. 2005. Purification and scale-up of a recombinant heavy chain fragment C of botulinum neurotoxin serotype E in Pichia pastoris GS115. Protein Expr Purif. In press
10. Edskes, H. K., V. T. Gray, and R. B. Wickner. 1999. The [URE3] prion is an aggregated form of Ure2p that can be cured by overexpression of Ure2p fragments. Proc Natl Acad Sci USA 96:1498-503.
11. Gomes-Solecki, M. J., D. R. Brisson, and R. J. Dattwyler. 2005. Oral vaccine that breaks the transmission cycle of the Lyme disease spirochete can be delivered via bait. Vaccine. In press.
12. Guay, H., R. L. Garcea, R. M. Welsh, and E. Szomolanyi-Tsuda. 2005. Murine model of polyomavirus (PyV) infection: antibody responses to PyV in normal and in partially immunodeficient mice, role of innate immune signals. J. Neurovirol. 11:95.
13. Harley, C. A., J. A. Holt, R. Turner, and D. J. Tipper. 1998. Transmembrane protein insertion orientation in yeast depends on the charge difference across transmembrane segments, their total hydrophobicity, and its distribution. J Biol Chem 273:24963-71.
14. Hepler, R. W., R. Kelly, T. B. McNeely, H. Fan, M. C. Losada, H. A. George, A. Woods, L. D. Cope, A. Bansal, J. C. Cook, G. Zang, S. L. Cohen, X. Wei, P. M. Keller, E. Leffel, J. G. Joyce, L. Pitt, L. D. Schultz, K. U. Jansen, and M. Kurtz. 2005. A recombinant 63-kDa form of Bacillus anthracis protective antigen produced in the yeast Saccharomyces cerevisiae provides protection in rabbit and primate inhalational challenge models of anthrax infection. Vaccine. In press.
15. Herre, J., S. Gordon, and G. D. Brown. 2004. Dectin-1 and its role in the recognition of beta-glucans by macrophages. Mol Immunol 40:869-76.
16. Hong, F., J. Yan, J. T. Baran, D. J. Allendorf, R. D. Hansen, G. R. Ostroff, P. X. Xing, N. K. Cheung, and G. D. Ross. 2004. Mechanism by which orally administered beta-1,3-glucans enhance the tumoricidal activity of antitumor monoclonal antibodies in murine tumor models. J Immunol 173:797-806.
17. Jamas, S., G. Ostroff, and D. J. Easson. 2000. Glucan drug delivery system and adjuvant. U.S. Pat. No. 5,741,495 World patent 0476063 PCT/US90/03440.
18. Kopecka, M., and M. Gabriel. 1992. The influence of congo red on the cell wall and (1-3)-beta-D-glucan microfibril biogenesis in Saccharomyces cerevisiae. Arch Microbiol 158:115-26.
19. Kortekangas-Savolainen, O., K. Kalimo, K. Lammintausta, and J. Savolainen. 1993. IgE-binding components of baker's yeast (Saccharomyces cerevisiae) recognized by immunoblotting analysis. Simultaneous IgE binding to mannan and 46-48 kD allergens of Saccharomyces cerevisiae and Candida albicans. Clin Exp Allergy 23:179-84.
20. Lambert, C., N. Thome, C. J. Kluck, and R. Prange. 2004. Functional incorporation of green fluorescent protein into hepatitis B virus envelope particles. Virology 330:158-67.
21. Lavelle, E. C. 2006. Lectins and microparticles for enhanced oral vaccination. Methods.
22. Madjid, M., S. Lillibridge, P. Mirhaji, and W. Casscells. 2003. Influenza as a bioweapon. J R Soc Med 96:345-6.
23. Mahnke, K., J. Knop, and A. H. Enk. 2003. Induction of tolerogenic DCs: ‘you are what you eat’. Trends Immunol 24:646-51.
24. Marcato, P., T. P. Griener, G. L. Mulvey, and G. D. Armstrong. 2005. Recombinant Shiga toxin B-subunit-keyhole limpet hemocyanin conjugate vaccine protects mice from Shigatoxemia. Infect Immun 73:6523-9.
25. Mayer, L., and L. Shao. 2004. Therapeutic potential of oral tolerance. Nat Rev Immunol 4:407-19. Meikle, P J, Binig, I, Hoogenraad N J, Clarke, A E, Stone, B A. 1991. The location of (1-3)-beta glucans in the walls of pollen tubes of Nicotinia alata using a (1-3)-beta glucan-specific monoclonal antibody. Planta 185: 1-8.
26. Navarro-Garcia, F., M. Pedroso, and R. Lopez-Revilla. 2000. Immunodulation of rat serum and mucosal antibody responses to Entamoeba histolytica trophozoites by beta-1,3-glucan and cholera toxin. Clin Immunol 97:182-8.
27. Nilsson, J., N. Miyazaki, L. Xing, B. Wu, L. Hammar, T. C. Li, N. Takeda, T. Miyamura, and R. H. Cheng. 2005. Structure and assembly of a T=1 virus-like particle in BK polyomavirus. J Virol 79:5337-45.
28. Ogra, P. L., H. Faden, and R. C. Welliver. 2001. Vaccination strategies for mucosal immune responses. Clin Microbiol Rev 14:430-45.
29. Ostroff, G. Unpublished observations.
30. Palucha, A., A. Loniewska, S. Satheshkumar, A. M. Boguszewska-Chachulska, M.
Umashankar, M. Milner, A. L. Haenni, and H. S. Savithri. 2005. Virus-like particles: models for assembly studies and foreign epitope carriers. Prog Nucleic Acid Res Mol Biol 80:135-68.
31. Pedersen, P. A., J. H. Rasmussen, and P. L. Joorgensen. 1996. Expression in high yield of pig alpha 1 beta 1 Na,K-ATPase and inactive mutants D369N and D807N in Saccharomyces cerevisiae. J Biol Chem 271:2514-22.
32. Ripaud, L., L. Maillet, and C. Cullin. 2003. The mechanisms of [URE3] prion elimination demonstrate that large aggregates of Ure2p are dead-end products. Embo J 22:5251-9.
33. Ross, E. D., H. K. Edskes, M. J. Terry, and R. B. Wickner. 2005. Primary sequence independence for prion formation. Proc Natl Acad Sci USA 102:12825-30.
34. Sasagawa, T., M. Tani, W. Basha, R. C. Rose, H. Tohda, Y. Giga-Hama, K. K. Azar, H.
Yasuda, A. Sakai, and M. Inoue. 2005. A human papillomavirus type 16 vaccine by oral delivery of LI protein. Virus Res 110:81-90.
35. Sasnauskas, K., A. Bulavaite, A. Hale, L. Jin, W. A. Knowles, A. Gedvilaite, A. Dargeviciute, D. Bartkeviciute, A. Zvirbliene, J. Staniulis, D. W. Brown, and R. Ulrich. 2002. Generation of recombinant virus-like particles of human and non-human polyomaviruses in yeast Saccharomyces cerevisiae. Intervirology 45:308-17.
36. Sheff, M. A., and K. S. Thorn. 2004. Optimized cassettes for fluorescent protein tagging in Saccharomyces cerevisiae. Yeast 21:661-70.
37. Shin, S. J., J. L. Bae, Y. W. Cho, D. Y. Lee, D. H. Kim, M. S. Yang, Y. S. Jang, and H. S. Yoo. 2005. Induction of antigen-specific immune responses by oral vaccination with Saccharomyces cerevisiae expressing Actinobacillus pleuropneumoniae ApxIIA. FEMS Immunol Med Microbiol 43:155-64.
38. Shin, S. J., S. W. Shin, E. J. Choi, D. Y. Lee, J. M. Ahn, M. S. Yang, Y. S. Jang, and H. S. Yoo. 2005. A predictive model for the level of sIgA based on IgG levels following the oral administration of antigens expressed in Saccharomyces cerevisiae. J Vet Sci 6:305-9.
39. Simon-Nobbe, B., G. Probst, A. V. Kajava, H. Oberkofler, M. Susani, R. Crameri, F. Ferreira, C. Ebner, and M. Breitenbach. 2000. IgE-binding epitopes of enolases, a class of highly conserved fungal allergens. J Allergy Clin Immunol 106:887-95.
40. Sloat, B. R., and Z. Cui. 2005. Strong Mucosal and Systemic Immunities Induced by Nasal Immunization with Anthrax Protective Antigen Protein Incorporated in Liposome-Protamine-DNA Particles. Pharm Res. In press.
41. Stubbs, A. C., K. S. Martin, C. Coeshott, S. V. Skaates, D. R. Kuritzkes, D. Bellgrau, A. Franzusoff, R. C. Duke, and C. C. Wilson. 2001. Whole recombinant yeast vaccine activates dendritic cells and elicits protective cell-mediated immunity. Nat Med 7:625-9.
42. Szomolanyi-Tsuda, E., J. D. Brien, J. E. Dorgan, R. L. Garcea, R. T. Woodland, and R. M. Welsh. 2001. Antiviral T-cell-independent type 2 antibody responses induced in vivo in the absence of T and NK cells. Virology 280:160-8.
43. Tegerstedt, K., A. V. Franzen, K. Andreasson, J. Joneberg, S. Heidari, T. Ramqvist, and T. Dalianis. 2005. Murine polyomavirus virus-like particles (VLPs) as vectors for gene and immune therapy and vaccines against viral infections and cancer. Anticancer Res 25:2601-8.
44. Vogel, M., J. Vorreiter, and M. Nassal. 2005. Quaternary structure is critical for protein display on capsid-like particles (CLPs): efficient generation of hepatitis B virus CLPs presenting monomeric but not dimeric and tetrameric fluorescent proteins. Proteins 58:478-88.
45. Wang, S., D. Heilman, F. Liu, T. Giehl, S. Joshi, X. Huang, T. H. Chou, J. Goguen, and S. Lu. 2004. A DNA vaccine producing LcrV antigen in oligomers is effective in protecting mice from lethal mucosal challenge of plague. Vaccine 22:3348-57.
46. Welsh, R. M., L. K. Selin, and E. Szomolanyi-Tsuda. 2004. Immunological memory to viral infections. Annu Rev Immunol 22:711-43. Wheeler, R. T. and Fink, G. R (2006). A drug-sensitive genetic network masks fungi from the immune system. PLoS pathogens 2: 328-339.
47. Willment, J. A., S. Gordon, and G. D. Brown. 2001. Characterization of the human beta-glucan receptor and its alternatively spliced isoforms. J Biol Chem 276:43818-23.
48. Wunderlich, G., and H. A. del Portillo. 2000. Biochemical and immunological properties of a viral hybrid particle expressing the Plasmodium vivax merozoite surface protein 1 C-terminal region. Mol Med 6:238-45.
49. Yamaguchi, M., K. Sugahara, K. Shiosaki, H. Mizokami, and K. Takeo. 1998. Fine structure of hepatitis B virus surface antigen produced by recombinant yeast: comparison with HBsAg of human origin. FEMS Microbiol Lett 165:363-7.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A yeast cell particle (YCP) having a reduced amount of mannan in its cell wall relative to that of a wild-type yeast, wherein the YCP comprises a heterologous antigen.

2. The YCP of claim 1, wherein a sufficient amount of mannan is removed to expose the underlying beta 1,3-glucan to allow it to interact with an M cell of the gastrointestinal tract of a eukaryote.

3. The YCP of claim 1, wherein about 10-50% of mannan is removed.

4. The YCP of claim 1, wherein the antigen is linked to a scaffold that allows the antigen to form an aggregate in the cytoplasm of a yeast cell.

5. The YCP of claim 4, wherein the scaffold is a protein that forms virus-like particles (VLPs).

6. The YCP of claim 5, wherein the scaffold is a VP1 capsid protein of mouse polyoma virus or a functional homolog thereof.

7. The YCP of claim 6, wherein the scaffold comprises SEQ ID NO: 20.

8. The YCP of claim 5, wherein the scaffold is a Hepatitis B surface antigen (HBsAg) or a functional homolog thereof.

9. The YCP of claim 8, wherein the scaffold comprises SEQ ID NO: 18.

10. The YCP of claim 4, wherein the scaffold is a non-pathogenic protein that self-aggregates in the cytoplasm of a yeast cell or a functional homolog thereof.

11. The YCP of claim 10, wherein the scaffold is non-pathogenic protein of yeast.

12. The YCP of claim 11, wherein the scaffold is a self-aggregating N-terminal portion of the yeast Ure2 protein or a functional homolog thereof.

13. The YCP of claim 12, wherein the scaffold comprises SEQ ID NO: 22.

14. The YCP of claim 4, wherein the antigen and the scaffold are linked through a linker.

15. The YCP of claim 14, wherein the linker is a flexible peptide linker.

16. The YCP of claim 15, wherein the linker comprises about 5-10 amino acids.

17. The YCP of claim 16, wherein the linker consists essentially of the amino acid sequence GGSSGGSS (SEQ ID NO: 23).

18. The YCP of claim 1, wherein the antigen is a protein from a pathogen or a functional homolog thereof.

19. The YCP of claim 18, wherein the antigen is selected from the group consisting of an LcrV protein from Yersinia pestis, a protective antigen (PA) from B. anthracis, hemagglutinin (HA) from influenza H5 and functional homologs thereof.

20. The method of claim 1, wherein the yeast is Saccharomyces cerevisiae.

21. A composition comprising a YCP of claim 1 and a pharmaceutically acceptable carrier or vehicle.

22. A vaccine preparation comprising a YCP of claim 1.

23. A nucleic acid comprising a nucleotide sequence encoding a fusion protein comprising an antigen and a scaffold that allows the antigen to form an aggregate in the cytoplasm of a yeast cell, wherein the nucleotide sequence encoding the fusion protein is operably linked to a promoter that is transcriptionally active in yeast.

24. The nucleic acid of claim 23, wherein the antigen is an antigen from a pathogen or a functional homolog thereof and the scaffold is a protein that forms VLPs, a non-pathogenic protein that self-aggregates in the cytoplasm of a yeast cell or a functional homolog thereof.

25. The nucleic acid of claim 24, wherein the scaffold is a self-aggregating N-terminal portion of the yeast Ure2 protein or a functional homolog thereof.

26. The nucleic acid of claim 24, wherein the antigen is selected from the group consisting of an LcrV protein from Yersinia pestis, a protective antigen (PA) from B. anthracis, hemagglutinin (HA) from influenza H5 and functional homologs thereof.

27. An expression vector comprising the nucleic acid of claim 23.

28. A yeast cell comprising the nucleic acid of claim 23.

29. The yeast cell of claim 28, which is S. cerevisiae yeast cell.

30. A method for preparing a yeast cell of claim 1, comprising
(i) providing a yeast cell comprising a heterologous antigen as an insoluble aggregate; and
(ii) subjecting the yeast cell to a treatment allowing sufficient removal of mannan from its outer cell wall layer to expose the underlying beta 1,3-glucan and allow it to interact with an M cell of the gastrointestinal tract of a eukaryote.

31. The method of claim 30, wherein step (ii) comprises incubating the yeast cell in a solution having a pH of about 10-13 at about 40-50° C. for about 5 to 10 minutes.

32. The method of claim 31, further comprising neutralizing the solution after step (ii).

33. A method for preparing a yeast cell of claim 1, comprising
(i) cultivating a yeast cell comprising a nucleic acid encoding a fusion protein comprising the heterologous antigen fused to a scaffold that allows the antigen to form an aggregate in the cytoplasm of the yeast cell, under conditions in which the yeast cell expresses the fusion protein; and
(ii) subjecting the yeast cell to a treatment allowing sufficient removal of mannan from its outer cell wall layer to expose the underlying beta 1,3-glucan and allow it to interact with an M cell of the gatrointestinal tract of a eukaryote.

34. The method of claim 33, wherein step (i) is preceded by a step in which the nucleic acid of step (i) is introduced into the yeast cell.

35. A method for preparing a vaccine, comprising combining a YCP of claim 1 with a pharmaceutically acceptable carrier.

36. A method for protecting a subject from an infection by a pathogen, comprising administering to a subject in need thereof a therapeutically effective dose of a YCP of claim 1, wherein the antigen is a protein from the pathogen or a functional homolog thereof that triggers a protective immune response against the pathogen.

37. The method of claim 36, wherein the YCP is administered orally.

38. The method of claim 37, for protection against plague, anthrax or influenza, wherein the antigen is selected from the group consisting of an LcrV protein from Yersinia pestis, a protective antigen (PA) from B. anthracis, hemagglutinin (HA) from influenza H5, respectively, and functional homologs thereof.

39. A method for treating a subject who has or is likely to develop a hyperproliferative disease, comprising administering to a subject in need thereof a therapeutically effective dose of a YCP of claim 1, wherein the antigen is a hyperproliferative-associated protein or a functional homolog thereof that triggers an immune response against the cells that cause the hyperproliferative disease.

40. A method for treating a subject who has or is likely to develop an autoimmune disease or allergy, comprising administering to a subject in need thereof a therapeutically effective dose of a YCP of claim 1, wherein the antigen is a protein associated with the autoimmune disease or allergy or a functional homolog thereof that triggers an immune response against the cells that cause the autoimmune disease or allergy.

41. A kit comprising one or more doses of YCPs of claim 1.


SEQUENCE LISTING (61 sequences)

SEQ ID NO: 1 - protein, 89 aa, Saccharomyces cerevisiae
MVDGNQMNNNKSRRNSSQRGNSNQRVNNQNENNFNGLAQSSNNNNSITTTFTNNNQINSQLNGINNNVNQTDQNVQNHGNSNENNSENL

SEQ ID NO: 2 - protein, 88 aa, Saccharomyces cerevisiae
MQSHQAESNSSQNGDQNGTNNLQNNRSNGINNFGNNRNQNNLESQRVNNTINNNKLNQFNGNNEVNNVQNQSSDNTNNNMSIVTTRNS

SEQ ID NO: 3 - protein, 89 aa, Saccharomyces cerevisiae
MNIRNQNQSTAVLNVNQQSNNGTSNSVNNLNFNNSGMQNHGRNFNQSTRNNNTNEKGGNNILNSNDERINNQQNQENNNTVDNSQNNSS

SEQ ID NO: 4 - protein, 89 aa, Saccharomyces cerevisiae
MMQRNGQQEGTNNNHSNINTQRNVFNNSANNNRNNNEGLNNNNSNFNNLVSNNQQVNVSSNSNINNQDNNKSILSGTSNDTTENRGQQQ

SEQ ID NO: 5 - protein, 89 aa, Saccharomyces cerevisiae
MNTNNSQGSFVDENQNRSIVKSRTVNMSQNNNTGNNNNAQLNNILNNTDSGHVSNNENRLGRQNNEFNQNSSQTNNGNNQQQSNNNNNI

SEQ ID NO: 6 - protein, 13 aa, Artificial Sequence
  Description of Artificial Sequence Synthetic peptide
GGTSGGSTGLSSG

SEQ ID NO: 7 - protein, 12 aa, Artificial Sequence
  Description of Artificial Sequence Synthetic peptide
LDGTSGGSGSSS

SEQ ID NO: 8 - protein, 14 aa, Artificial Sequence
  Description of Artificial Sequence Synthetic peptide
GGTSGGSTGLESSG

SEQ ID NO: 9 - protein, 13 aa, Artificial Sequence
  Description of Artificial Sequence Synthetic peptide
GGSSGGSSGLDSS

SEQ ID NO: 10 - DNA, 717 bp, Artificial Sequence
  Description of Artificial Sequence Synthetic construct
atgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcacttatggtgttcaatgcttttcaagatacccagatcatatgaaacagcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagctaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataa

SEQ ID NO: 11 - protein, 238 aa, Artificial Sequence
  Description of Artificial Sequence Synthetic construct
MSKGEELFTGVVPILVELDGDVNGHKFSVSGEGEGDATYGKLTLKFICTTGKLPVPWPTLVTTFTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKANFKIRHNIEDGSVQLADHYQQNTPIGDGPVLLPDNHYLSTQSALSKDPNEKRDHMVLLEFVTAAGITHGMDELYK

SEQ ID NO: 12 - protein, 10 aa, Artificial Sequence
  Description of Artificial Sequence Synthetic peptide
GGGGSGGGGT

SEQ ID NO: 13 - DNA, 1212 bp, Artificial Sequence
  Description of Artificial Sequence Synthetic construct
atggatatcgatccttataaagaattcggagctactgtggagttactctcgtttctcccgagtgacttctttccttcagtacgagaccttctggataccgccagcgcgctgtatcgggaagccttggagtctcctgagcactgcagccctcaccatactgccctcaggcaagcaattctttgctggggggagctcatgactctggccacgtgggtgggtgttaacctcgaggatggtggaggtggctccggagggggtggtaccatgagcaagggcgaggaactgttcactggcgtggtcccaattctcgtggaactggatggcgatgtgaatgggcacaaattttctgtcagcggagagggtgaaggtgatgccacatacggaaagctcaccctgaaattcatctgcaccactggaaagctccctgtgccatggccaacactggtcactaccctcacctatggcgtgcagtgcttttccagatacccagaccatatgaagcagcatgactttttcaagagcgccatgcccgagggctatgtgcaggagagaaccatctttttcaaagatgacgggaactacaagacccgcgctgaagtcaagttcgaaggtgacaccctggtgaatagaatcgagctgaagggcattgactttaaggaggatggaaacattctcggccacaagctggaatacaactataactcccacaatgtgtacatcatggccgacaagcaaaagaatggcatcaaggtcaacttcaagatcagacacaacattgaggatggatccgtgcagctggccgaccattatcaacagaacactccaatcggcgacggccctgtgctcctcccagacaaccattacctgtccacccagtctgccctgtctaaagatcccaacgaaaagagagaccacatggtcctgctggagtttgtgaccgctgctgggatcacacatggcatggacgagctgtacaagggtggaggtggctccggagggggtggatctagagacctggtagtcagttatgtcaacactaatatgggtttaaagttcaggcaactcttgtggtttcacattagctgcctcactttcggccgagaaacagttatagaatatttggtgtctttcggagtgtggatcagaactcctccagcttataggcctccgaatgcccctatcctgtcgacactcccggagactacggtagta

SEQ ID NO: 14 - protein, 404 aa, Artificial Sequence
  Description of Artificial Sequence Synthetic construct
MDIDPYKEFGATVELLSFLPSDFFPSVRDLLDTASALYREALESPEHCSPHHTALRQAILCWGELMTLATWVGVNLEDGGGGSGGGGTMSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYKGGGGSGGGGSRDLVVSYVNTNMGLKFRQLLWFHISCLTFGRETVIEYLVSFGVWIRTPPAYRPPNAPILSTLPETTVV

SEQ ID NO: 15 - DNA, 558 bp, Hepatitis B virus
atggacatcgacccttataaagaatttggagcttctgcggagttactctcgtttttgccttctgacttctttccttccgtccgggatctactagacacagccaaagctttgtttcaggaagccttagagtctcctgagcattgttcgcctcaccatactgcactcaggcaagcaattctttgctggggggacctaatgactctagctacctgggtgggtgctaatttggaagatccagcttctagagacctagtagtcaattatgtcaacactactgcgggcctaaagttcagacaactcttgtggtttcacatttcttgtctcacttttggaagagaaacagtgatagagtatttggtgtctttcggagtgtggattcgcactcctccaccttatagaccaccaaatgcccctatcttatcaacacttccggaaactactgttgttagataccgagaccgaggcaggtccactagaagaagaactccctcgcctcgcagacgaagatctcaatcgccgcgtcgcagaagatctcaatctcgggaatctcaatgttag

SEQ ID NO: 16 - protein, 185 aa, Hepatitis B virus
MDIDPYKEFGASAELLSFLPSDFFPSVRDLLDTAKALFQEALESPEHCSPHHTALRQAILCWGDLMTLATWVGANLEDPASRDLVVNYVNTTAGLKFRQLLWFHISCLTFGRETVIEYLVSFGVWIRTPPPYRPPNAPILSTLPETTVVRYRDRGRSTRRRTPSPRRRRSQSPRRRRSQSRESQC

SEQ ID NO: 17 - DNA, 678 bp, Artificial Sequence
  Description of Artificial Sequence Synthetic construct
atggagaacatcacatcaggattcctaggacccctgctcgtgttacaggcggggtttttcttgttgacaagaatcctcacaataccgcagagtctagactcgtggtggacttctctcaattttctatggggatctcccgtgtgtcttggccaaaattcgcagtccccaacctccaatcactcaccaacctcctgtcctccaatttgtcctggttatcgctggatgtgtctgcggcgttttatcatattcctcttcatcctgctgctatgcctcatcttcttattggttcttctggattatcaaggtatgttgcccgtttgtcctctaattccaggatcaacaacaaccagtacgggaccatgcaaaacctgcacgactcctgctcaaggcaactctatgtttccctcatgttgctgtacaaaacctacggatggaaattgcacctgtattcccatcccatcgtcctgggctttcgcaaaatacctatgggagtgggcctcagtccgtttctcttggctcagtttactagtgccatttgttcagtggttcgtagggctttcccccactgtttggctttcagctatatggatgatgtggtattgggggccaagtctgtacagcatcgtgagtccctttataccgctgttaccaattttcttttgtctttgggtatacatt

SEQ ID NO: 18 - protein, 226 aa, Artificial Sequence
  Description of Artificial Sequence Synthetic construct
MENITSGFLGPLLVLQAGFFLLTRILTIPQSLDSWWTSLNFLWGSPVCLGQNSQSPTSNHSPTSCPPICPGYRWMCLRRFIIFLFILLLCLIFLLVLLDYQGMLPVCPLIPGSTTTSTGPCKTCTTPAQGNSMFPSCCCTKPTDGNCTCIPIPSSWAFAKYLWEWASVRFSWLSLLVPFVQWFVGLSPTVWLSAIWMMWYWGPSLYSIVSPFIPLLPIFFCLWVYI

SEQ ID NO: 19 - DNA, 1172 bp, Artificial Sequence
  Description of Artificial Sequence Synthetic construct
ctcgagaagatggcccccaaaagaaaaagcggcgtctctaaatgcgagacaaaatgtacaaaggcctgtccaagacccgcacccgttcccaaactgcttattaaagggggtatggaggtgctggaccttgtgacagggccagacagtgtgacagaaatagaagcttttctgaaccccagaatggggcagccacccacccctgaaagcctaacagagggagggcaatactatggttggagcagagggattaatttggctacatcagatacagaggattccccagaaaataatacacttcccacatggagtatggcaaagctccagcttcccatgctcaatgaggacctcacctgtgacaccctacaaatgtgggaggcagtctcagtgaaaaccgaggtggtgggctctggctcactgttagatgtgcatgggttcaacaaacccacagatacagtaaacacaaaaggaatttccactccagtggaaggcagccaatatcatgtgtttgctgtgggcggggaaccgcttgacctccagggacttgtgacagatgccagaacaaaatacaaggaagaaggggtagtaacaatcaaaacaatcacaaagaaggacatggtcaacaaagaccaagtcctgaatccaattagcaaggccaagctggataaggacggaatgtatccagttgaaatctggcatccagatccagcaaaaaatgagaacacaaggtactttggcaattacactggaggcacaacaactccacccgtcctgcagttcacaaacaccctgacaactgtgctcctagatgaaaatggagttgggcccctctgtaaaggagagggcctatacctctcctgtgtagatataatgggctggagagttacaagaaactatgatgtccatcactggagagggcttcccagatatttcaaaatcaccctgagaaaaagatgggtcaaaaatccctatcccatggcctccctcataagttcccttttcaacaacatgctcccccaagtgcagggccaacccatggaaggggagaacacccaggtagaggaggttagagtgtatgatgggactgaacctgtaccgggggaccctgatatgacgcgctatgttgaccgctttggaaaaacaaagactgtatttcctggaaattaagatctgcc

SEQ ID NO: 20 - protein, 384 aa, Artificial Sequence
  Description of Artificial Sequence Synthetic construct
MAPKRKSGVSKCETKCTKACPRPAPVPKLLIKGGMEVLDLVTGPDSVTEIEAFLNPRMGQPPTPESLTEGGQYYGWSRGINLATSDTEDSPENNTLPTWSMAKLQLPMLNEDLTCDTLQMWEAVSVKTEVVGSGSLLDVHGFNKPTDTVNTKGISTPVEGSQYHVFAVGGEPLDLQGLVTDARTKYKEEGVVTIKTITKKDMVNKDQVLNPISKAKLDKDGMYPVEIWHPDPAKNENTRYFGNYTGGTTTPPVLQFTNTLTTVLLDENGVGPLCKGEGLYLSCVDIMGWRVTRNYDVHHWRGLPRYFKITLRKRWVKNPYPMASLISSLFNNMLPQVQGQPMEGENTQVEEVRVYDGTEPVPGDPDMTRYVDRFGKTKTVFPGN

SEQ ID NO: 21 - DNA, 201 bp, Artificial Sequence
  Description of Artificial Sequence Synthetic construct
atgatgaataacaacggcaaccaagtgtcgaatctctccaatgcgctccgtcaagtaaacataggaaacaggaacagtaatacaaccaccgatcaaagtaatataaattttgaattttcaacaggtgtaaataataataataataacaatagcagtagtaataacaataatgttcaaaacaataacagcggccgctcgagc

SEQ ID NO: 22 - protein, 67 aa, Artificial Sequence
  Description of Artificial Sequence Synthetic construct
MMNNNGNQVSNLSNALRQVNIGNRNSNTTTDQSNINFEFSTGVNNNNNNNSSSNNNNVQNNNSGRSS

SEQ ID NO: 23 - protein, 8 aa, Artificial Sequence
  Description of Artificial Sequence Synthetic peptide
GGSSGGSS

SEQ ID NO: 24 - protein, 354 aa, Saccharomyces cerevisiae
MMNNNGNQVSNLSNALRQVNIGNRNSNTTTDQSNINFEFSTGVNNNNNNNSSSNNNNVQNNNSGRNGSQNNDNENNIKNTLEQHRQQQQAFSDMSHVEYSRITKFFQEQPLEGYTLFSHRSAPNGFKVAIVLSELGFHYNTIFLDFNLGEHRAPEFVSVNPNARVPALIDHGMDNLSIWESGAILLHLVNKYYKETGNPLLWSDDLADQSQINAWLFFQTSGHAPMIGQALHFRYFHSQKIASAVERYTDEVRRVYGVVEMALAERREALVMELDTENAAAYSAGTTPMSQSRFFDYPVWLVGDKLTIADLAFVPWNNVVDRIGINIKIEFPEVYKWTKHMMRRPAVIKALRGE

SEQ ID NO: 25 - protein, 7 aa, Artificial Sequence
  Description of Artificial Sequence Synthetic peptide
GQQNNYN

SEQ ID NO: 26 - DNA, 37 bp, Artificial Sequence
  Description of Artificial Sequence Synthetic oligonucleotide
ggcagatctcattaatttccaggaaatacagtctttg

SEQ ID NO: 27 - DNA, 48 bp, Artificial Sequence
  Description of Artificial Sequence Synthetic oligonucleotide
  CDS (6)..(47)
gctgctgcggccgctcgagcatgagtaaaggagaagaacttttcactg

SEQ ID NO: 28 - protein, 14 aa, Artificial Sequence
  Description of Artificial Sequence Synthetic peptide
CGRSSMSKGEELFT

SEQ ID NO: 29 - DNA, 44 bp, Artificial Sequence
  Description of Artificial Sequence Synthetic oligonucleotide
catggcatggatgaactatacaaataagatctgaattcagcagc

SEQ ID NO: 30 - DNA, 44 bp, Artificial Sequence
  Description of Artificial Sequence Synthetic oligonucleotide
gctgctgaattcagatcttatttgtatagttcatccatgccatg

SEQ ID NO: 31 - DNA, 20 bp, Artificial Sequence
  Description of Artificial Sequence Synthetic oligonucleotide
gatcgtcgacggatccccag

SEQ ID NO: 32 - DNA, 63 bp, Artificial Sequence
  Description of Artificial Sequence Synthetic oligonucleotide
  CDS (3)..(56)
caaagactgtatttcctggaaatctagacggaactagtggtggaagtgggtcgacggtcctgg

SEQ ID NO: 33 - protein, 18 aa, Artificial Sequence
  Description of Artificial Sequence Synthetic peptide
KTVFPGNLDGTSGGSGST

SEQ ID NO: 34 - DNA, 63 bp, Artificial Sequence
  Description of Artificial Sequence Synthetic oligonucleotide
ccaggaccgtcgacccacttccaccactagttccgtctagatttccaggaaatacagtctttg

SEQ ID NO: 35 - DNA, 67 bp, Artificial Sequence
  Description of Artificial Sequence Synthetic oligonucleotide
  CDS (1)..(60)
ggcatggatgaactatacaaaggaggtactagtggtggaagtactggtctagagtcgacggtcctgg

SEQ ID NO: 36 - protein, 20 aa, Artificial Sequence
  Description of Artificial Sequence Synthetic peptide
GMDELYKGGTSGGSTGLEST

SEQ ID NO: 37 - DNA, 67 bp, Artificial Sequence
  Description of Artificial Sequence Synthetic oligonucleotide
ccaggaccgtcgactctagaccagtacttccaccactagtacctcctttgtatagttcatccatgcc

SEQ ID NO: 38 - DNA, 15 bp, Artificial Sequence
  Description of Artificial Sequence Synthetic oligonucleotide
  CDS (1)..(15)
ggccgctcgagcatg

SEQ ID NO: 39 - protein, 5 aa, Artificial Sequence
  Description of Artificial Sequence Synthetic peptide
GRSSM

SEQ ID NO: 40 - DNA, 12 bp, Artificial Sequence
  Description of Artificial Sequence Synthetic oligonucleotide
ggctcgagaatg

SEQ ID NO: 41 - DNA, 42 bp, Artificial Sequence
  Description of Artificial Sequence Synthetic oligonucleotide
ggatgcgtcgacgctagcagaatgatgaataacaacggcaac

SEQ ID NO: 42 - DNA, 37 bp, Artificial Sequence
  Description of Artificial Sequence Synthetic oligonucleotide
  CDS (1)..(27)
caaaacaataacagcggccgctcgagaattcagcagc

SEQ ID NO: 43 - protein, 9 aa, Artificial Sequence
  Description of Artificial Sequence Synthetic peptide
QNNNSGRSR

SEQ ID NO: 44 - DNA, 37 bp, Artificial Sequence
  Description of Artificial Sequence Synthetic oligonucleotide
gctgctgaattctcgagcggccgctgttattgttttg

SEQ ID NO: 45 - DNA, 30 bp, Artificial Sequence
  Description of Artificial Sequence Synthetic oligonucleotide
gggctcgagaatggagaacatcrcatcagg

SEQ ID NO: 46 - DNA, 33 bp, Artificial Sequence
  Description of Artificial Sequence Synthetic oligonucleotide
cttttgtctttgggtatacatttaagatctccc

SEQ ID NO: 47 - DNA, 33 bp, Artificial Sequence
  Description of Artificial Sequence Synthetic oligonucleotide
gggagatcttaaatgtatacccaaagacaaaag

SEQ ID NO: 48 - DNA, 37 bp, Artificial Sequence
  Description of Artificial Sequence Synthetic oligonucleotide
  CDS (16)..(36)
gggagcactagtaagatggatatcgatccttataaag

SEQ ID NO: 49 - protein, 7 aa, Artificial Sequence
  Description of Artificial Sequence Synthetic peptide
MDIDPYK

SEQ ID NO: 50 - DNA, 14 bp, Artificial Sequence
  Description of Artificial Sequence Synthetic oligonucleotide
tcgacgactagtcg

SEQ ID NO: 51 - DNA, 14 bp, Artificial Sequence
  Description of Artificial Sequence Synthetic oligonucleotide
tcgacgagtactcg

SEQ ID NO: 52 - DNA, 44 bp, Artificial Sequence
  Description of Artificial Sequence Synthetic oligonucleotide
  CDS (1)..(30)
ctgtcgacactcccggagactacggtagtataagatctcgcgcc

SEQ ID NO: 53 - protein, 10 aa, Artificial Sequence
  Description of Artificial Sequence Synthetic peptide
LSTLPETTVV

SEQ ID NO: 54 - DNA, 44 bp, Artificial Sequence
  Description of Artificial Sequence Synthetic oligonucleotide
ggcgcgagatcttatactaccgtagtctccgggagtgtcgacag

SEQ ID NO: 55 - DNA, 60 bp, Artificial Sequence
  Description of Artificial Sequence Synthetic oligonucleotide
  CDS (10)..(60)
cggcgagccactagtggtggaagtggttcgtcgaccatgattagagcctacgaacaaaac

SEQ ID NO: 56 - protein, 17 aa, Artificial Sequence
  Description of Artificial Sequence Synthetic peptide
TSGGSGSSTMIRAYEQN

SEQ ID NO: 57 - DNA, 36 bp, Artificial Sequence
  Description of Artificial Sequence Synthetic oligonucleotide
gctagatgacacgtctggtaaataagatctcgcgcc

SEQ ID NO: 58 - DNA, 36 bp, Artificial Sequence
  Description of Artificial Sequence Synthetic oligonucleotide
ggcgcgagatcttatttaccagacgtgtcatctagc

SEQ ID NO: 59 - protein, 6 aa, Artificial Sequence
  Description of Artificial Sequence Synthetic peptide
SSTSAA

SEQ ID NO: 60 - DNA, 34 bp, Artificial Sequence
  Description of Artificial Sequence Synthetic oligonucleotide
cggctctagactcgaggaagatggcccccaaaag

SEQ ID NO: 61 - DNA, 37 bp, Artificial Sequence
  Description of Artificial Sequence Synthetic oligonucleotide
caaagactgtatttcctggaaattaatgagatctgcc